(19) United States
(12) Patent Application Publication
(10) Pub. No.: US 2024/0000702 A1
(43) Pub. Date: Jan. 4, 2024

(54) METHOD AND MATERIAL FOR DETECTING AND INFLUENCING THE ABSORPTION AND/OR RELEASE OF BIOACTIVE SUBSTANCES USING ELECTRICALLY CONDUCTIVE HYDROGELS

(71) Applicant: LEIBNIZ-INSTITUT FÜR POLYMERFORSCHUNG DRESDEN E.V., 01069 Dresden (DE)

(72) Inventors: Teuku Fawzul Akbar, Dresden (DE); CHRISTOPH TONDERA, Leipzig (DE); IVAN MINEV, Ruse (BG); UWE FREUDENBERG, Dresden (DE); CARSTEN WERNER, Dresden (DE)

(73) Assignee: LEIBNIZ-INSTITUT FÜR POLYMERFORSCHUNG DRESDEN E.V., 01069 Dresden (DE)

(21) Appl. No.: 18/039,186

(22) PCT Filed: Nov. 11, 2021
(86) PCT No.: PCT/DE2021/100902

§ 371 Date: May 26, 2023

(30) Foreign Application Priority Data
Nov. 27, 2020 (DE) 10 2020 131 547.3

(51) Int. Cl. A61K 9/00; C12N 5/00; A61K 9/06; A61K 47/32; H01B 1/12

(52) U.S. Cl. CPC A61K 9/0009; C12N 5/0068; A61K 9/06; A61K 47/32; H01B 1/122

(57) ABSTRACT

A method for detecting and influencing an uptake of bioactive substances in a hydrogel material and/or release of bioactive substances from the hydrogel material, wherein the hydrogel material is a polymer network formed from charged and uncharged building blocks, the affinity of which network for bioactive substances is configurable by parameters defining the charged building blocks, and whose electrical charge storage capacity depend on interaction with the hydrogel binding of bioactive substances to the hydrogel material. When the hydrogel material is contacted with a biofluid, a change in electrical resistance and/or a change in charge storage capacity of the hydrogel material is detected. Uptake or a release of bioactive substances from the hydrogel material into the biofluid is determined by change in electrical resistance. The invention further relates to a suitable electrically conductive hydrogel material.

The invention relates to a method for influencing and detecting an absorption of bioactive substances in a hydrogel material and/or a release of bioactive substances from the hydrogel material. Furthermore, the invention relates to an electrically conductive hydrogel material which is suitable for carrying out the method.

Substance release systems and substance absorption systems based on hydrogels are promising for applications in biotechnology and in particular in medicine, since hydrogels are similar in their water content and mechanical properties to human physiological tissue in comparison with many other biomaterials and allow an inclusion of various substances and their targeted release. By definition, hydrogels are strongly hydrated, covalently or physically crosslinked polymers which allow substances to be reversibly bound to affine polymer building blocks of the hydrogel network via various, non-covalent interactions and thus either to remove the substances selectively from a biofluid or a living tissue, i.e. to sequester them in the hydrogel, or to release them from the hydrogel to the biofluid or the living tissue. Such substances can be protein-based signal molecules from the class of cytokines, chemokines, hormones, neurotransmitters, growth factors or non-protein-based low molecular active ingredients, so-called small molecules, which take over one or more biological functions of the abovementioned protein-based signal molecules or also non-protein-based chemical/medical active ingredients such as antimicrobial active ingredients, antiseptics, dyes which are reversibly linked to the affinity-mediating polymer building blocks either via charge interactions and/or hydrophobic Interactions or other specific chemical interactions such as hydrogen bridge bonds in the hydrogel. Hydrogel systems are known from the prior art in which the physicochemical properties of a hydrogel for sequestration and/or release for certain substances must be determined already during formation. This is achieved by means of a specific charge characteristic of the hydrogel network, such as is known, for example, from WO 2018/162009 A2, or by the targeted setting of physical properties, such as mesh width. It is also known to release covalently coupled substances in a controlled manner by enzymatic triggers, light or variable pH values, wherein no reversible substance binding is possible to the disadvantage of such hydrogel networks. A controlled sequestration of substances and thus depletion from a biofluid or a tissue environment is thus not possible.

In the known systems, which reversibly realize the affinity for the substances via non-covalent interactions, the affinity and thus the control of the sequestration or release of substances is determined by their intrinsic network architecture, i.e., which is impressed during the formation of the hydrogel network. Subsequent modulation is only possible via the aforementioned external triggers light, pH value changes or enzymatic cleavage.

Apart from intrinsically charged hydrogels, electrically conductive hydrogels are described in biomedical research. They are predominantly used as a coating for electrode materials, for example for neural electrodes, in order to optimize their biocompatibility and charge injection. Due to their mechanical similarity to tissues and organs, use of soft hydrated polymer materials leads to lower repulsion reactions compared to the use of metal electrodes. In addition, in classical metal electrodes, the charge injection, which is essential for the stimulation of tissues and organs, takes place only at the surface of the electrodes. Nanostructuring attempts are made to increase the surface and thus the contact area between the electrode and the tissue fluid (biofluid), which results in increased charge injection. Since the biofluid can penetrate into the entire volume of the material in the case of electrically conductive hydrogels, the contact area is already significantly larger without further structuring than in the case of metal electrodes of comparable size. This also leads to a significantly increased charge injection. As a measure of the ability to transfer charges into physiological solutions, the term charge storage capacity is used in literature.

Conductive substances such as carbon nanotubes and/or electrically conductive polymers are used for the preparation of such conductive hydrogels, wherein the conductive substance is usually distributed in a hydrogel precursor solution and the hydrogel is then formed by polymerization and crosslinking in a further component which is independent of the conductive substance. In this case, the overarching property is that the electrically conductive component is usually hydrophobic and therefore does not form a hydrogel.

Due to their diverse application possibilities and their simple and cost-effective processing, conductive organic polymers are increasingly used in industrial applications. The polymers work according to the principle of semiconductors. The most important part is a conjugated π-electron system which extends through the entire polymer backbone. The intrinsic electric conductivity of these polymers is low. In order to create free charge carriers with the aim of increasing the molecular electrical conductivity, charged molecules are required which remove electrons from the polymer system (p-doping) or introduce electrons into the polymer system (n-doping). This process is called primary doping. The most commonly used method is p-doping. The doping and the resulting interaction of hydrophobic polymers with highly charged hydrophilic molecules additionally reduces the hydrophobicity of the polymer-doping complexes and thus permits the use of these polymers in aqueous solutions or hydrogels.

Apart from the primary doping, the arrangement of the conductive polymer chains relative to one another is also of decisive importance. For the transport of charge carriers over longer distances, these must be transmitted between the electrically conductive polymer chains. For this purpose, a uniform distribution of the polymer chains in the volume with simultaneous spatial proximity to one another is necessary. Aqueous solutions of conductive polymers and the particular doping molecule usually form suspensions. The lack of interaction of the conductive polymer chains with one another results in a low electrical conductivity. By the structural reorganization of the conductive polymer chains relative to one another (secondary doping), the necessary spatial proximity of the polymer chains relative to one another can be achieved and the conductivity can be further increased as a result. This can be achieved, for example, by increasing the ionic strength and the associated shielding effects of the charge (characterized over the Debye length).

An example with the conductive poly-3,4-ethylenedioxythiophene (PEDOT) is described by Zhenan Bao and other authors (DOI: 10.1038/s41467-018-05222-4, and US20190390068A1). As is known from this document, a weakly crosslinked hydrogel is first formed from a mixture of hydrophilic polystyrene sulfonate (PSS) and PEDOT (primary doping) by physical loops of the PSS and using ionic liquids (secondary doping), which hydrogel is then mechanically stabilized by formation of a pseudo-interpenetrating network formed by radical crosslinking reaction of acrylate monomers. After the polymerization, an electrically conductive hydrogel network results (10.1038/s41467-018-05222-4, and US20190390068A1), wherein the secondary polymer network merely serves to stabilize and improve the mechanical properties of the material. The electrical properties of the hydrogel result solely from the primary, non-covalent PEDOT:PSS hydrogel.

A further method of obtaining electrically conductive hydrogels is known from U.S. Pat. No. 9,299,476 B2. According to the method known from U.S. Pat. No. 9,299,476 B2, a biopolymer-based hydrogel network carrying functional anionic groups is formed on the surface of an electrode and deposited. The primary network thus obtained is swollen or converted into a solution containing 3,4-ethylenedioxythiophene (EDOT) monomers and an electrical polymerization of the EDOT monomers to give poly-3,4-ethylenedioxythiophene (PEDOT) is initiated by the influence of an electrical voltage. A pseudo-interpenetrating polymer network is formed by the ionic interaction of the PEDOT with the anionic functional groups of the primary network. These anionic groups simultaneously have a p-doping effect on the PEDOT (primary doping) and, in combination, create an electrically conductive hydrogel material. Due to the predetermined distribution of the anionic groups covalently bonded in the hydrogel and the subsequent polymerization of the PEDOT around the primary hydrogel network, hydrogels having high electrical conductivity can be obtained even without secondary doping because of the uniform distribution of the PEDOT polymer chains.

The previously known hydrogel materials permit sequestration and/or release of substances by means of the intrinsically impressed physicochemical characteristic or, in the case of electrically conductive hydrogel materials, show limited modulability and specificity for the interaction with certain bioactive substances.

It is an object of the invention to propose a method by which concentrations of bioactive substances in a hydrogel material or in an environment of a hydrogel material can be influenced and determined. Further, it is an object of the invention to provide an electrically conductive hydrogel material which can be modulated with respect to its electrical properties and physicochemical properties and permits a reversible sequestration and/or release in particular of substances carrying positively charged groups. At the same time, the binding of substances by resulting characteristic changes in the electrical properties should be detectable with this material.

The object is achieved by a method with the features according to claim 1 and an electrically conductive hydrogel material with the features of claim 7. Further developments are indicated in the respective dependent claims. Uses of the electrically conductive hydrogel material are indicated in claims 19 to 22.

The invention comprises a method for detecting and influencing an absorption of bioactive substances in a hydrogel material and/or a release of bioactive substances from the hydrogel material, wherein the hydrogel material by definition is a polymer network formed from anionically charged building blocks and uncharged building blocks, whose affinity for bioactive substances can be configured on the basis of parameters defining the anionically charged building blocks and has an electrically conductive component whose electrical resistance and electrical charge storage capacity depends on an interaction with hydrogel building blocks and a binding of bioactive substances to the hydrogel material, wherein the electrically conductive component is suitable to change the anionic charge of the hydrogel material and its affinity for bioactive substances by the influence of an electrical potential. In the method, the predefined hydrogel material is brought into contact with a biofluid, wherein a change in the electrical resistance and/or a change in the charge storage capacity of the hydrogel material is detected and an absorption of bioactive substances in the hydrogel material or a release of bioactive substances from the hydrogel material into the biofluid is determined on the basis of the detected change in the electrical resistance and/or the detected charge storage capacity change, and/or wherein a concentration of bioactive substances in the biofluid and/or a concentration of bioactive substances in the hydrogel material is influenced by an electrical potential acting on the hydrogel material.

The polymer network formed from anionically charged building blocks and uncharged building blocks is an anionically charged polymer network. The anionically charged polymer network can be configured on the basis of parameters which define the anionically charged building blocks.

The hydrogel material according to the definition, which is electrically conductive, is referred to below as hydrogel material for the sake of simplicity.

In the sense of the invention, biofluids are understood to be physiological solutions, cell cultures and living tissue. The instructions for contacting the hydrogel material with a biofluid can therefore be understood as contacting by immersion in a physiological solution or as surface contacting of the hydrogel material with a living tissue in vivo and in vitro.

In the sense of the invention, bioactive substances are understood to be protein-based and non-protein-based bioactive substances, active substances and “small molecules” which have signal properties of cytokines, chemokines, hormones, neurotransmitters or growth factors and cause further biological effects. Bioactive substances can be, in particular, pharmaceutical active ingredients. A molecular weight of less than or equal to 70 kDa Is considered to be the overarching feature for all above-mentioned bioactive substances.

The method according to the invention comprises detecting an absorption of bioactive substances in a hydrogel material and/or a release of bioactive substances from the hydrogel material, as well as influencing an absorption of bioactive substances in a hydrogel material and/or a release of bioactive substances from the hydrogel material. It is thus possible to detect and influence bioactive substances or, according to the alternative, to detect or influence bioactive substances.

The fact that a change in the electrical resistance of the hydrogel material and/or an electrical charge capacity change of the hydrogel material is/are detected for detecting an absorption of bioactive substances in the hydrogel material and/or a release of bioactive substances from the hydrogel material is based on the finding that the change in the electrical resistance of the hydrogel material and/or an electrical charge capacity change of the hydrogel material is influenced by the binding of bioactive substances to the hydrogel material. Thus, the electrical resistance of the hydrogel material is increased as a result of the sequestration and binding to or in the hydrogel material, wherein the electrical conductivity of the hydrogel material decreases as a result of the loading of the anionic groups with bioactive substances. Conversely, the electrical resistance of the hydrogel material is reduced when bioactive substances are dissolved from the hydrogel material, wherein the electrical conductivity of the hydrogel material increases.

The charge storage capacity is calculated by measuring the cyclic voltammetry. For this purpose, the current flow between the working electrode (hydrogel material) and the counter electrode (carbon electrode) is measured in a 3-electrode structure, while the applied electrical potential of −0.6 to 0.8 V (potential between working electrode and Ag/AgCl reference electrode) is varied in 5 cyclic passes. The scan rate is 50 mV/s. Subsequently, the negative part of the area under the curve is integrated (MultiTrace 4.3, PalmSens 4) and the charge storage capacity is calculated from this with the following formula:

charge storage capacity currentI*voltageU scan−rate

As an integral, the software outputs the value I*U in the unit [A][V]. [A]=[C/s] applies. The division of the scan rate [V/s] results in the charge [C] which is transmitted by the material. This is then set in the ratio to the volume, since the interface to the surrounding medium relates to the complete volume of the hydrogel and thus a calculation of the surface/contact area is not possible.

In order to detect an absorption of bioactive substances in the hydrogel material and/or a release of bioactive substances from the hydrogel material, it is alternatively possible to determine a change in the electrical conductivity of the hydrogel material, wherein an absorption of bioactive substances in the hydrogel material and/or a release of bioactive substances from the hydrogel material is determined on the basis of the change in the electrical conductivity. The binding of bioactive substances in the electrically conductive hydrogel material takes place via non-covalent interactions of the bioactive substances with the polymer chains of the hydrogel material. In this context, ionic interactions play an important role. The doping of the conductive polymer is changed by the interaction of anionic and cationic groups of the bound bioactive substances with the polymer network of the hydrogel material carrying anionic groups and with the cationic, electrically conductive polymer. The anionic groups in the bound substances can contribute to doping and increase the conductivity. Cationic groups, on the other hand, compensate for negative charges of the anionic polymer component and thus have a negative influence on the doping. In addition, hydrophobic regions of the bioactive substances can influence the interaction of the PEDOT polymer chains with one another. Depending on the molecular type of the bound bioactive substances and the concentration of the bioactive substances, a specific change in the electrical properties of the hydrogel material thus takes place.

An absorption and/or a release of bioactive substances can be understood as a change in the concentration of bioactive substances in the hydrogel material. Further, it can be provided that a discrete concentration of a bioactive substance is assigned to a discrete conductivity value, a discrete electrical resistance value or a discrete value of the electrical charge storage capacity.

The electrical resistance of the hydrogel material can be determined as a direct current resistance or as an impedance. A frequency range from 0.01 Hz to 1 MHz can be predetermined for impedance detection. It can be provided that, in order to detect a binding of bioactive substances to or in the hydrogel, a change in the impedance of the hydrogel material at at least one frequency in the range from 0.1 Hz to 1 MHz is measured. A change in the charge storage capacity can also be taken into account.

In order to influence a concentration of bioactive substances in the biofluid and/or a concentration of bioactive substances in the hydrogel material, an electrical potential is applied to the hydrogel material. Due to the influence of an electrical potential on the hydrogel material, the anionic charge of the hydrogel material and its affinity for bioactive substances are changed, so that a binding of bioactive substances in or on the hydrogel material is influenced as a result of a change in potential.

A release of bioactive substances from the hydrogel material presupposes that a bioactive substance is bound to or in the hydrogel material before contact with the biofluid. It can therefore be provided that, for the release of bioactive substances, the hydrogel material is charged electrically or chemically with a predetermined concentration of the predetermined bioactive substance before contacting it with the biofluid.

According to an embodiment variant of the method according to the invention, it is provided that the polymer network can be configured in its composition on the basis of at least three parameters defining the anionically charged building blocks, selected from a group of parameters P0, P1, P2, P3, wherein parameter P0 corresponds to a value from the number of the ionized, anionic groups, assuming 30% ionization of all anionic groups, per unit volume, of the hydrogel material swollen under physiological conditions, parameter P1 corresponds to a value from the number of the strongly anionic groups, with an intrinsic pK_{S} value of less than 2.5, per unit volume of the hydrogel material swollen under physiological conditions, parameter P2 corresponds to a value from the number of the strongly anionic groups, with an intrinsic pK_{S} value of less than 2.5, per repeat unit divided by the molar mass of the repeat unit, and parameter P3 corresponds to a value for describing the amphiphilia of the anionic building blocks, wherein an electric resistance and/or an electric charge storage capacity of the hydrogel material is predetermined by parameter values of a parameter configuration of the hydrogel material. A detailed definition of the parameter values and their determination can be found in the description below.

An interaction of bioactive substances can furthermore be based on the substance-specific value Pp, which is calculated from the ratio of the net charge of a bioactive substance and the surface of the bioactive substance accessible to water. For protein-based substances, any protein structure is available in the Protein Data Bank (PDB, http://www.rcsb.org/). The net charge of the selected protein structure is calculated with the Delphi Web Server (http://compbio.clemson.edu/sapp/delphi_webserver/) with default settings at pH 7. The protein surface accessible to the solvent water is calculated using the PyMol software (www.pymol.org) using a solvent radius of water of 1.4 Å. Then Pp is calculated from the net charge divided by the protein surface accessible to the solvent water multiplied by a factor of 1000000 to obtain the unit 10⁻⁶×[1/A² or A⁻²]. For non-protein-based substances, the net charge which can be derived from the chemical structure and which corresponds to the excess of anionic or cationic groups, and the molecular surface which is accessible by the solvent water and which was derived in analogy to the formation rule for parameter P3 by using the ChemDraw19.0 and ChemAxon MarvinSketch 19.21 software, are calculated. The value obtained is multiplied by a factor of 1000000 to obtain the unit 10⁻⁶×[1/A² or A⁻²].

Loading of the hydrogel material, i.e. immobilization of a predetermined concentration of a bioactive substance in or on the hydrogel material, can be effected in different ways. According to a first method, which can also be referred to as the first loading method, a predetermined concentration of the bioactive substance or substances intended for binding is mixed with the anionically charged hydrogel building blocks and integrated into the polymer network. In the subsequent formation of the electrically conductive hydrogel materials by incorporation of the electrically conductive component, the bioactive substances are then already contained in the polymer network according to the predetermined concentration. In this procedure, it is advantageous that the entire amount of the bioactive substance or of the bioactive substances is contained quantitatively in the hydrogel material after the formation of the polymer network. In other words, the loading of the bioactive substance or of the bioactive substances takes place independently of the parameters P0, P1, P2, P3 and independently of the substance-specific value Pp. The reaction conditions prevailing in the formation of hydrogel material may have an unfavorable influence on the structure of the bioactive substances, for which reason not all bioactive substances are suitable for immobilization according to the first loading method.

According to a second method, which can also be referred to as a second loading method, the electrically conductive hydrogel material is formed with a predetermined parameter configuration and predetermined parameter values and the electrically conductive hydrogel material is brought into contact with an aqueous solution or a biofluid as a loading solution with a predetermined substance concentration for a predetermined period of time. In this case, the bioactive substances in solution are absorbed from the aqueous solution or the biofluid into the hydrogel material and bound as a function of the parameters P0, P1, P2, P3. Finally, the loaded hydrogel material is removed from the loading solution. The bioactive substances immobilized on or in the hydrogel material can then be released by the influence of an electrical potential on an environment, preferably a biofluid or a living tissue. Due to the influence of the electrical potential, there is also the possibility that bioactive substances from the environment of the hydrogel material are sequestered into the hydrogel material.

According to a third method of loading, which can also be referred to as the third loading method, a predetermined electrically conductive hydrogel material which has a predetermined parameter configuration with predetermined parameter values is brought into contact with an aqueous solution or a biofluid as a loading solution with a predetermined substance concentration for a predetermined period of time and is thereby exposed to the influence of an electrical potential. In this case, bioactive substances are absorbed (sequestered) from the loading solution into the hydrogel material as a function of the predetermined parameter values. It may be necessary to maintain the potential acting on the hydrogel material in order to keep the binding of the bioactive substances constant. Otherwise, if the potential or the current is changed, the bioactive substances can be released from the hydrogel material. It is essential here that the sequestration or release of bioactive substances is based not only on the influence of an electrical potential but also on an affinity of the hydrogel material for certain bioactive substances which is predetermined by the parameters P0, P1, P2, P3, Pp.

It has been found that a structure formation and distribution of the electrically conductive component in the hydrogel material which influences the electrical conductivity of the hydrogel material can be influenced by the parameter values of a parameter configuration. It can therefore be provided that a hydrogel material is predetermined which has a predetermined electrical conductivity on the basis of a predetermined parameter configuration with predetermined parameter values.

In the method, it can further be provided that the hydrogel material for the absorption of bioactive substances is exposed to an electrical potential, compared to an Ag/AgCl reference electrode, in the range from 1 mV to 1000 mV, preferably in the range from 400 mV to 600 mV. For the release of bioactive substances, the hydrogel material can be charged with an electric potential, compared to an Ag/AgCl reference electrode, in the range from −1 mV to −1000 mV, preferably in a range from −400 mV to −600 mV.

The hydrogel material can be charged with a constant electric current of greater than 0 mA for the absorption of bioactive substances, wherein the direction of the electric current flow is changed for the release of bioactive substances.

The invention also comprises an electrically conductive hydrogel material which is suitable for carrying out the method described above. The hydrogel material according to the invention has a polymer network formed from anionically charged building blocks and uncharged building blocks or consists of such polymer network, which can be configured in its composition on the basis of at least three parameters defining the anionically charged building blocks, selected from a group of parameters P0, P1, P2, P3, wherein parameter P0 corresponds to a value from the number of the ionized, anionic groups, assuming 30% ionization of all anionic groups, per unit volume of the hydrogen material swollen under physiological conditions, parameter P1 corresponds to a value from the number of the strongly anionic groups, with an intrinsic pK_{S} value of less than 2.5, per unit volume of the hydrogen material swollen under physiological conditions, parameter P2 corresponds to a value from the number of the strongly anionic groups, with an intrinsic pK_{S} value of less than 2.5, per repeat unit divided by the molar mass of the repeat unit, and parameter P3 corresponds to a value for describing the amphiphilia of the anionic building blocks. Furthermore, the hydrogel material has an electrically conductive component which is incorporated into the polymer network, wherein an electrical conductivity, an electrical resistance and/or an electrical charge storage capacity of the hydrogel material is predeterminable by parameter values of a parameter configuration of the hydrogel material.

Parameters P0 to P3 are defined in more detail on the basis of the following definitions and formation rules:
Parameter P0: the value for P0, which can be indicated in μmol/ml, corresponds to 30% of the total number of anionic groups based on the hydrogel volume swollen under physiological conditions (0.154 mmol/l NaCl, pH buffered to 7.4). The calculation can be calculated, for example, from the polymer concentration of the hydrogel building blocks with swelling in physiological solution.
Parameter P1: the value for P1, which can be indicated in μmol/ml, corresponds to the number of strongly anionic groups which have a pKs value of less than 2.5, based on the hydrogel volume swollen under physiological conditions (0.154 mmol/l NaCl, pH buffered to 7.4).
Parameter P2: the value for P2, which can be indicated in mmol/(g/mol), corresponds to the number of strongly anionic groups with a pKs value of less than 2.5 per anionically charged building block divided by the respective molecular weight of the anionic building block.
Parameter P3: Parameter P3, which describes the amphiphilia of the anionic building blocks, is calculated by dividing the distribution coefficient octanol/water (Log P value) of the anionic building block by the surface of the anionic building block accessible to the solvent water. This can be done as follows with the help of the ChemDraw19.0 and ChemAxon MarvinSketch 19.21 software: Using the software ChemDraw19.0, each anionic building block with a length of the polymer residue of 22 carbon atoms or, in the case of sugar-based structures with a total of 2 disaccharide units, is represented as a complete chemical structure. By using the software application ChemAxon MarvinSketch 19.21, the distribution coefficient octanol/water (Log P value) Is then calculated by reading in the structural formulas represented by ChemDraw19.0 and the surface accessible by the solvent water is calculated with a solvent radius of 1.4 Λ. The value obtained is multiplied by a factor of 1000 to obtain the unit 10⁻³×[1/A² or A⁻²].

The hydrogel material according to the invention is an essential part for carrying out the method according to the invention. The features relating to the hydrogel according to the invention can therefore be used for a more detailed explanation of the method according to the invention, in particular for defining the hydrogel material, and vice versa. The hydrogel material according to the invention is based on a polymer network carrying anionic groups which is defined in its properties by the parameters P0, P1, P2, P3. With the help of these parameters P0 to P3, the formation of a (pseudo) interpenetrating network of electrically conductive components and thus the electrical properties of the electrically conductive hydrogel material which forms are controlled. Advantageously, the charge properties of the electrically conductive hydrogel material can be continuously modulated by applying an electrical potential or by the influence of an electrical potential, wherein the modulation of the electrical charge properties in connection with the parameters P0, P1, P2, P3 controls an affinity of the electrically conductive hydrogel material to bioactive substances and thus the depletion of substances from a biofluid in contact with the conductive hydrogel (sequestration) or the release of bioactive substances from the hydrogel material into the biofluid can be regulated continuously, reversibly and in real time in direct dependence on the applied electrical potential.

By using different charged building blocks carrying anionic groups and varying their concentration in the hydrogel material and the number and density of strongly anionic groups along the polymer chain of the uncharged building blocks, it is possible to synthesize hydrogel materials of different configurations with the same degree of crosslinking and solids content according to the parameters P0-P3. In order to create electrically conductive hydrogel materials, the polymer PEDOT is preferably chemically polymerized around the primary hydrogel networks as an electrically conductive component as a pseudo-interpenetrating network. The hydrogel materials obtained differ in their electrical properties and in the sequestration and release of bioactive substances as a function of the configured parameters or parameter values and their constellation.

Parameter P0, which describes all the anionic groups which are actually ionized independently of their intrinsic pKa in the swollen hydrogel, exerts a somewhat smaller influence on the electrical properties of the resulting hydrogel material in comparison with parameter P1. The weakly anionic groups contained in parameter P0 do not represent an effective doping for PEDOT, as a result of which the electrical conductivity or the electrical resistance is influenced in comparison with parameter P1, which describes the strongly acidic groups with a pKa<2.5. The integral number of strongly negatively charged anionic groups (P1), on the other hand, directly influences the electrical conductivity or the electrical resistance. The minimum electrical conductivity is achieved with a completely undoped hydrogel material (for P1=0) or without an electrically conductive component. The higher the number of highly charged anionic groups in the hydrogel material, the higher the electrical conductivity and the lower the electrical resistance. The cause is the generation of the free charge carriers by the p-doping of the PEDOT. The larger the number of doping units, the more free charge carriers can be formed. For the local charge density of the strongly anionic groups (P2), a direct influence on the electrical properties can likewise be expected. The reduction of the local charge density with constant P0 and P1 leads to a reduction of the electrical conductivity of the hydrogel materials. The reason is the local electrostatic interaction of the negatively charged polymers with the positively charged PEDOT polymer chains. An insufficient local negative charge leads to a weaker interaction of the positively charged PEDOT polymer chains with the anionic polymer or the anionically charged building blocks. This makes doping more difficult, which leads to lower electrical conductivity. Even with a moderate value for P1 and too low a value for P2, this presumably does not permit increased electrical conductivity to undoped hydrogel materials to be observed. The interaction between the amphiphilic anionic polymer and the hydrophobic electrically conductive polymer (PEDOT) can likewise be configured by parameter P3. Increased hydrophobicity of the anionically charged building blocks makes it possible to generate increased electrical conductivities even at a significantly lower P1 value. The reason is the high affinity of the hydrophobic PEDOT units to the hydrophobic groups on the anionically charged building block, which has a positive influence on the doping. In addition, the hydrophobic groups already facilitate a better penetration of the monomer units (EDOT) into the polymer network during the PEDOT synthesis and thus a better incorporation of the PEDOT chains.

For voltage-dependent, respectively current-dependent sequestration and release of substances, the integral (P0 or P1) and local charge density (P2) play a predominant role in the various applied voltages/currents in the entire hydrogel material. Without applied voltage, the positive charge of the PEDOT compensates for part of the negative charges of the anionically charged modules. As a result, in comparison to hydrogel materials which consist of a polymer network without an electrically conductive component, negative charged molecules can also be bound to a greater extent. Depending on the affinity of the bioactive substances to the anionic polymer, i.e. to the anionically charged building block, the binding of positively charged bioactive substances is likewise possible. By configuring the parameters P0, P1, P2, P3, the ratio of the positive charges of the PEDOT and the anionic groups of the charged module can be set under constant PEDOT polymerization conditions. In this way, sequestration (absorption) of positively or negatively charged bioactive substances can be made possible. By applying a voltage, the charge of the PEDOT of neutral to 66% of positive charges per monomer unit can additionally be set. As a result, the binding can additionally be set in a continuous manner. The release of the bound bioactive substances can likewise be set as a function of the configured parameters P0, P1, P2, P3 of the polymer network in combination with the charge of the PEDOT, i.e. the electrically conductive component. In addition, a continuous configuration can also be effected by setting the charge of the PEDOT by applying an electric voltage or an electric current. As a result, a reduced or increased release of bioactive substances of different charges can be set continuously.

The anionically charged building blocks may be selected from a group containing poly(acrylic acid-co-4-acrylamidomethylbenzenesulfonic acid), poly(acrylic acid-co-acrylamidoethanesulfonic acid), poly(acryilc acid-co-acrylamidoethane hydrogen sulfate), poly(4-styrene sulfonic acid-co-maleic acid), sulfated glycosaminoglycans, in particular heparin, selectively desulfated heparin derivatives, heparan sulfate, chondrol sulfate, keratan sulfate and dermatan sulfate. The uncharged building blocks may be polymers or crosslinker molecules containing amino groups or thiol groups with at least two amino groups or thiol groups, wherein the charged and uncharged building blocks are crosslinked to the polymer network, obtainable by activating carboxyl groups of poly(acrylic acid-co-4-acrylamidomethylbenzenesulfonic acid) and/or poly(acrylic acid-co-acrylamidoethanesulfonic acid) and/or poly(acrylic acid-co-acrylamidoethane hydrogen sulfate) and/or poly(4-styrene sulfonic acid-co-maleic acid) monoacid), and/or sulfated glycosaminoglycans, in particular heparin, and/or selectively desulfated heparin derivatives and/or heparan sulfate, and/or chondrotin sulfate and/or keratan sulfate and/or dermatan sulfate with EDC/sulfo-NHS and either a direct crosslinking with the polymers or the crosslinker molecules containing amino groups with the at least two amino groups in each case with amide formation or a functionalization of the activated carboxyl groups by means of bifunctional crosslinker molecules, which in each case contain an amino group and a group capable of Michael type addition, and the subsequent crosslinking with the polymers or the crosslinking molecules containing thiol groups with the at least two thiol groups in each case via a Michael type addition.

The electrical impedance of the hydrogel material according to the invention is variable in the range from 150Ω to 10, measured at a frequency of 0.01 ΩHz. The parameter values of a parameter configuration are preferably selected such that an electrical impedance of preferably 30Ω is achieved. The preferred impedance value of 30Ω is achieved when a parameter configuration with the parameters P1, P2, P3 is predetermined for P1=110 μmol/ml, for P2=4.5 mmol/(g/mol) and for P3=−5.9 A⁻². A further advantageous impedance value of 63Ω is achieved if a parameter configuration with parameters P1, P2, P3 is predetermined for P1=12 μmol/ml, for P2=4.5 mmol/(g/mol) and for P3=6.8 A⁻².

The electric charge storage capacity of the hydrogel material according to the invention can be varied in the range from 900 mC/ml to 4000 mC/ml. Preferably, the parameter values of a parameter configuration are selected such that a charge storage capacity of 3040 mC/ml is achieved. The preferred charge storage capacity of 2480 mC/ml is achieved when a parameter configuration with the parameters P1, P2, P3 is predetermined for P1=110 μmol/ml, for P2=4.5 mmol/(g/mol) and for P3=−5.9 A⁻². A further advantageous charge storage capacity of also 3040 mC/ml is achieved if a parameter configuration with parameters P1, P2, P3 is predetermined for P1=12 μmol/ml, for P2=4.5 mmol/(g/mol) and for P3=6.8 A⁻².

The electrically conductive component may be a Π-conjugated, electrically conductive polymer or a polymer composition of polypyrrole, polyaniline, polythiophene and/or poly(3,4-ethylenedoxothiophene) (PEDOT).

According to a further development of the conductive hydrogel according to the invention, it can be provided that the polymers containing amine and thiol groups are selected as uncharged building blocks from the class of polyethylene glycols (PEG), poly(2-oxazolines) (POX), polyvinylpyrrolidones (PVP), polyvinyl alcohols (PVA) and/or polyarylamides (PAM), and that the crosslinker molecules containing amine or thiol groups are non-polymeric, bifunctional crosslinker molecules.

It can further be provided that polymers with conjugated enzymatically cleavable peptides which have either lysine or cysteine as reactive amino acid in the peptide sequence can be used as uncharged building blocks for polymer network formation. The enzymatically cleavable peptides can be cleavable by human or bacterial proteases, in particular MMPs, cathepsins, elastases, aureolysin and/or blood coagulation enzymes.

According to a further advantageous development of the hydrogel material according to the invention, it can be provided that bioactive and/or antiadhesive molecules with an amino group or carboxyl group and/or cell-Instructive peptides are attached to the hydrogel network under formation of a covalent bond via lysine or cysteine in a sequence on the charged building blocks poly(acrylic acid-co-4-acrylamidomethylbenzenesulfonic acid) and/or poly(acrylic acid-co-acrylamidoethanesulfonic acid) and/or poly(acrylic acid-co-acrylamidoethanesulfonic acid) and/or poly(4-styrene sulfonic acid-co-maleic acid), and/or glycosaminoglycans such as heparin and/or selectively desulfated heparin derivatives and/or heparan sulfate and/or chondrotin sulfate and/or keratan sulfate and/or dermatan sulfate or on derivatives thereof with groups capable of Michael type addition. The bioactive molecules can be antimicrobial substances, for example antibiotics or antiseptics, or pharmaceutical active ingredients.

The electrically conductive hydrogel material is preferably predetermined with a parameter configuration with the parameters P0, P2, P3 or with the parameters P1, P2, P3. The parameter values can be varied in predetermined parameter value ranges. The value for the parameter P0 can be predetermined in a range from 0 to 80 μmol/ml, the value for the parameter P1 can be predetermined in a range from 0 to 150 μmol/ml, the value for the parameter P2 can be predetermined in a range from 0 to 10 mmol/(g/mol) and the value for the parameter P3 can be predetermined in a range from −7×10⁻³ to 7×10³ A⁻².

The hydrogel material according to the invention can preferably have a storage module of 0.2 kPa to 22 kPa.

A use of the hydrogel material according to the invention can be provided for factor management in vivo for controlling angiogenesis, in immune diseases, cancers, diabetes, neurodegenerative diseases, Crohn's disease, ulcerative colitis, multiple sclerosis, asthma, rheumatoid arthritis or cutaneous wound healing and bone regeneration. A further use or application of the electrically conductive hydrogel material is the electrical stimulation of cells or tissues.

Furthermore, the hydrogel material according to the invention can be used for the targeted purification of proteins from cell lysates of microbial or eukaryotic origin.

A use of the hydrogel material according to the invention can also be provided for the in vitro cell culture and organ culture of induced pluripotent stem cells (iPS-), as well as further stem cells and precursor cells not to be assigned iPS, primary cells obtained from patients, immortalized cell lines, as well as heart tissue, muscle tissue, kidney tissue, liver tissue and nerve tissue.

The core of the invention is the provision of the hydrogel according to the invention which, as the most important feature under physiological conditions (ionic strength and pH value), carries anionic, that is to say negatively charged, groups. The physicochemical properties of the hydrogel material according to the invention, in particular the (anionic) charge characteristic which is decisive for the intrinsic affinity to substances and the characteristic of the chemical environment of the anionic groups which is decisive for non-ionic interactions, are described via the following parameters P0, P1, P2 und P3. In addition to the properties already described, physical properties such as swelling, stiffness and mesh size of the hydrogel material according to the invention can also be changed or influenced by the influence of an electrical potential. The presence of covalently bound signal units can be graded over wide ranges and largely independently of the parameters P0-P3.

In order to produce the hydrogel material according to the invention, a predetermined native polymer network which has a predetermined parameter configuration with predetermined parameter values is swollen in a solution in which the electrically conductive component is contained and the polymerization or crosslinking is then initiated to an electrically conductive polymer network. The native polymer network with its anionic groups thereby acts doping on the conductive component via electrostatic interactions. According to the invention, the produced hydrogel material consists of one or more conductive polymer systems which interact with the predetermined native polymer network by physical interactions. Surprisingly, it has been found that the structure and distribution of the electrically conductive component can be influenced by the parameters P0-P3 and, associated therewith, the electrical properties of electrical resistance, electrical conductivity and electrical charge storage capacity and also the mechanical and physicochemical properties of the hydrogel material. Due to the influence of an electrical potential, i.e. by applying a voltage to the electrically conductive component, the charge properties of the polymer network and thus the affinity to bioactive substances are continuous, reversible and can be modulated in real time.

Further advantageous properties result from the surprising influences by electrostimulation of cells with the help of the hydrogel material according to the invention in combination with the release or sequestration of bioactive substances. Furthermore, the mechanical properties of the material can also be varied in real time as a function of the parameters P0 to P3 via the modulation of the current flow.

The production of the hydrogel material according to the invention is explained in more detail below with reference to an exemplary embodiment: For the electrical functionalization of the hydrogel material, the conductive polymer poly-3,4-ethylenedioxythiophene (PEDOT) is polymerized as an electrically conductive component around the predetermined native polymer network. For this purpose, the completely swollen polymer network is first incubated for 3 hours at room temperature in a solution consisting of 0.4 M ammonium peroxodisulfate (APS) dissolved in 1 M HCl. Incubation is then carried out for 6 hours at room temperature in 0.4 M 3,4-ethylenedioxythiophene (EDOT) in mineral oil. During the second incubation step, an oxidative polymerization of PEDOT takes place by the native polymer network. An SPH-PEDOT is obtained, which is then washed in mineral oil, hexane and PBS. As a result of the method used, no electrodes are necessary in comparison with the electrodeposition of PEDOT. In addition, any desired volume bodies can be made electrically conductive.

Immediately after oxidative polymerization, PEDOT possesses an inherent positive charge (see 10.1021/acs.jpcb.9b01745, 10.1021/acsapm.8b00061). Due to the non-covalent interactions between PEDOT and the anionically charged building blocks of the hydrogel and the non-covalent interaction between the individual PEDOT chains (probably mostly hydrophobic interactions), a pseudo-Interpenetrating network is formed between the anionically charged polymer network and PEDOT. At the same time, the negative charges of the anionically charged polymer network function as doping for PEDOT. Depending on the integral and local charge density, which are defined by the parameters P0 or P1 and P2, the doping degree of the PEDOT changes. This has a direct influence on the electrical properties of the conductive hydrogel material. Since PEDOT is a hydrophobic polymer, the distribution and crosslinking of the PEDOT chains among themselves is strongly dependent on the hydrophobicity of the environment, which is defined by the parameter P3, which likewise has an influence on the electrical properties of the hydrogel material.

In addition to releasing charged molecules, conductive hydrogels having different electrical properties can be obtained by the changes in the doping degree as a result of predetermined values of the P1 and P2 and the hydrophobicity P3. By means of a high doping, hydrogel materials having a high conductivity can be obtained. These can be used as biocompatible electrodes. Due to the mechanical properties of hydrogel materials, which are very similar to biological tissue, foreign body reactions of an organism can be reduced compared to SPH-PEDOT electrodes in comparison to classical metal electrodes. In addition, in classical metal electrodes, the charge transfer for the electrical stimulation takes place only at the contact surface from the metal to the tissue (physiological solution). Due to the distribution of the conductive component within the hydrogel material and the possibility that physiological liquids can diffuse into the hydrogel material, a charge transfer can take place within the volume of the hydrogel material. Advantageously, lower electrical voltages are required for the same charge injection, which results in a lower heat development and consequently a less tissue damage.

The hydrogel material according to the invention, in combination with the method according to the invention, permits a sustained release of active substances into a biofiuid or living tissue.

Further details, features and advantages of designs of the invention will become apparent from the following description of example embodiments with reference to the associated drawings and tables. The following is shown:

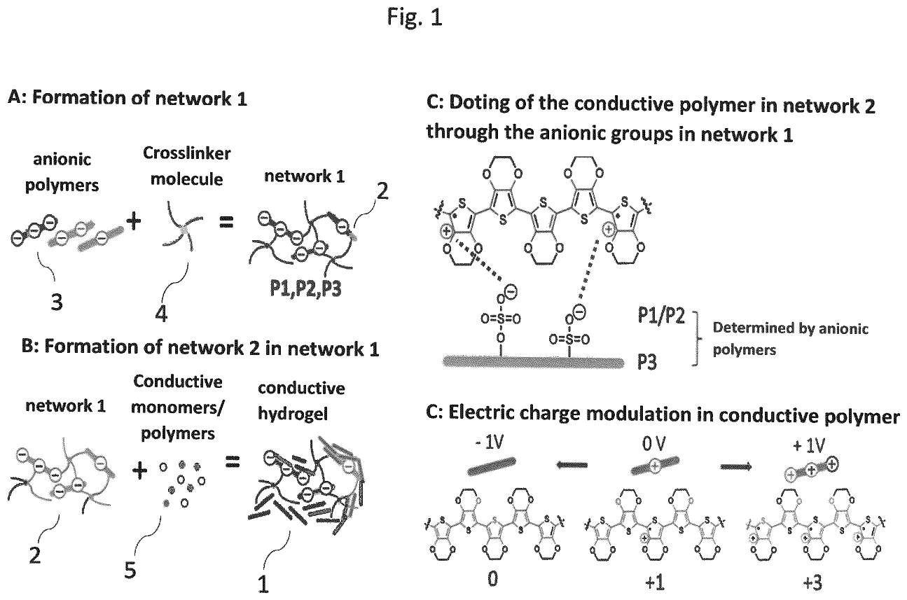

FIG. 1: a schematic representation for explanation of the hydrogel material according to the invention,

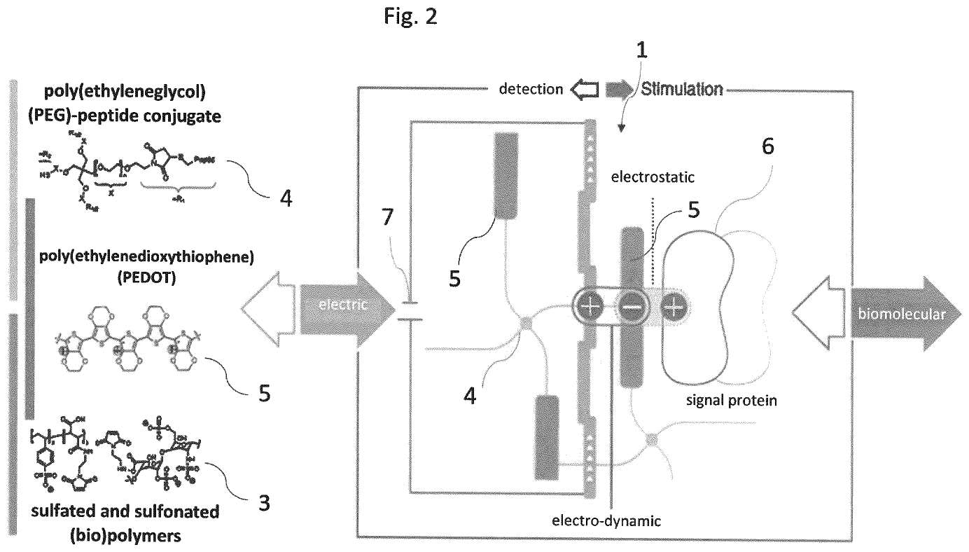

FIG. 2: a further schematic representation for further explanation of the invention,

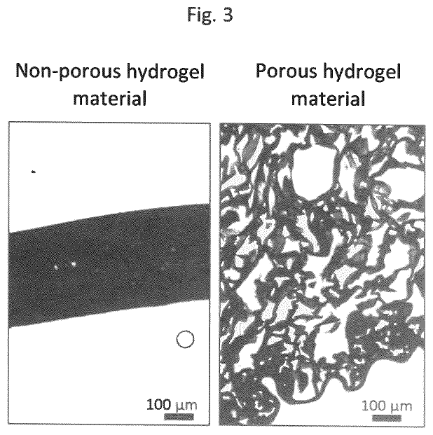

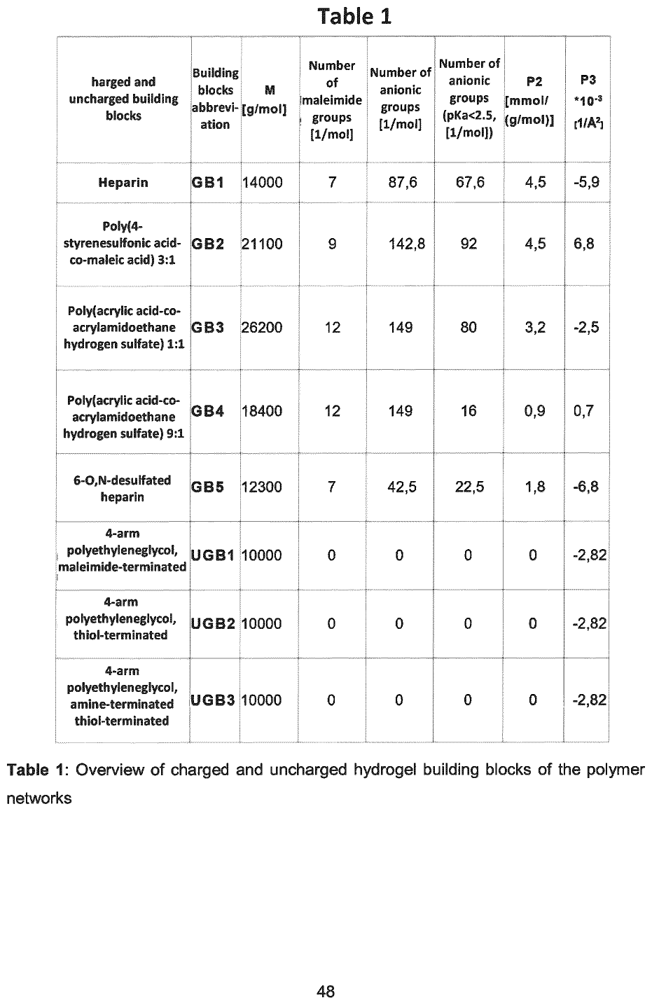

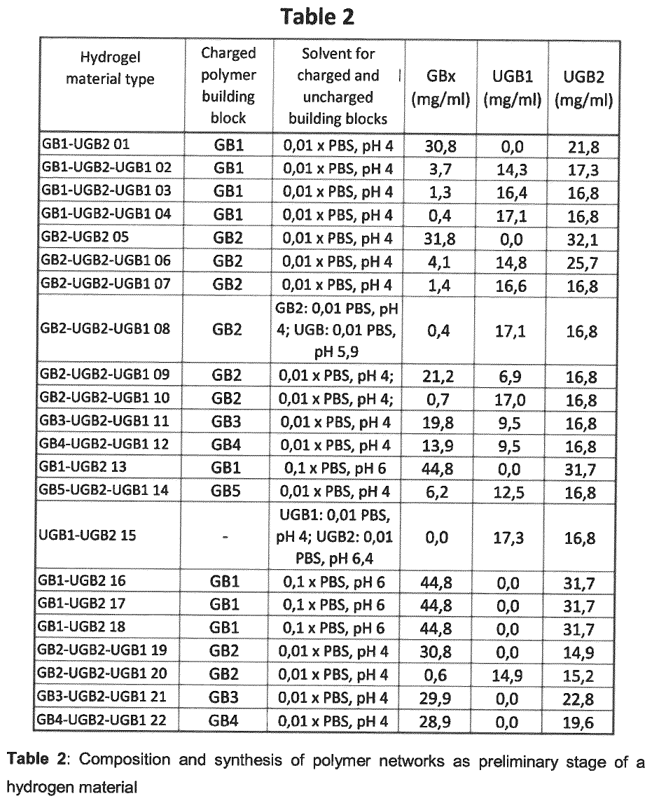

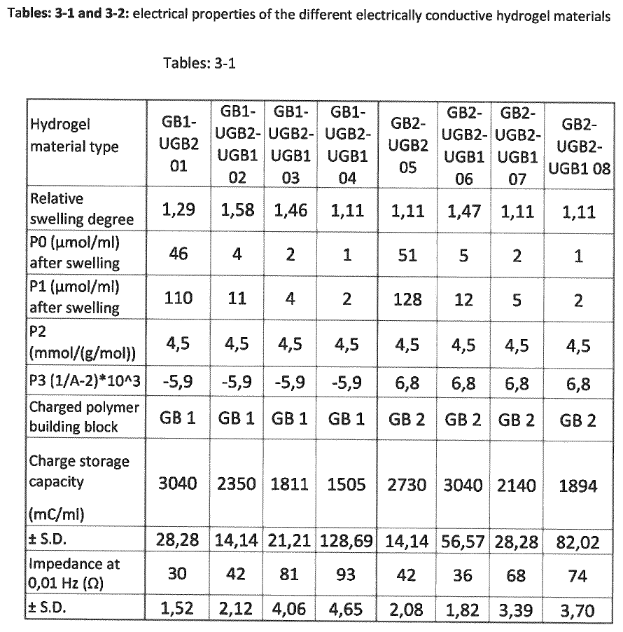

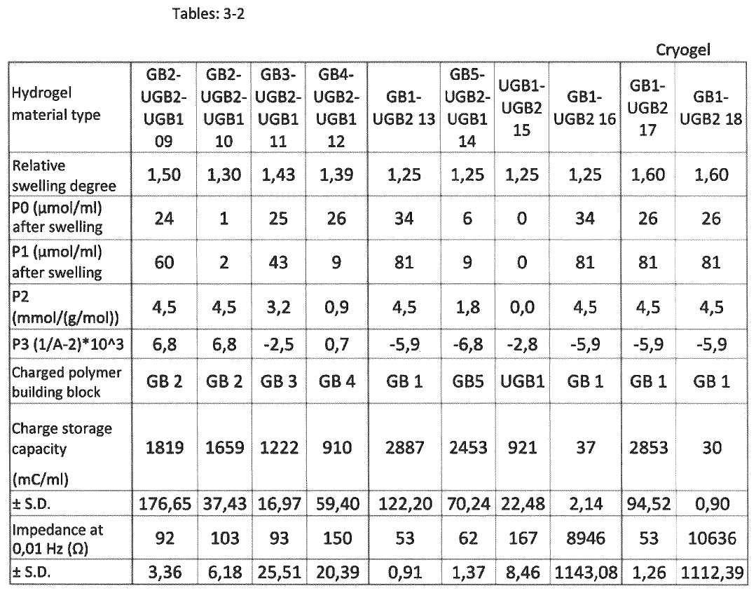

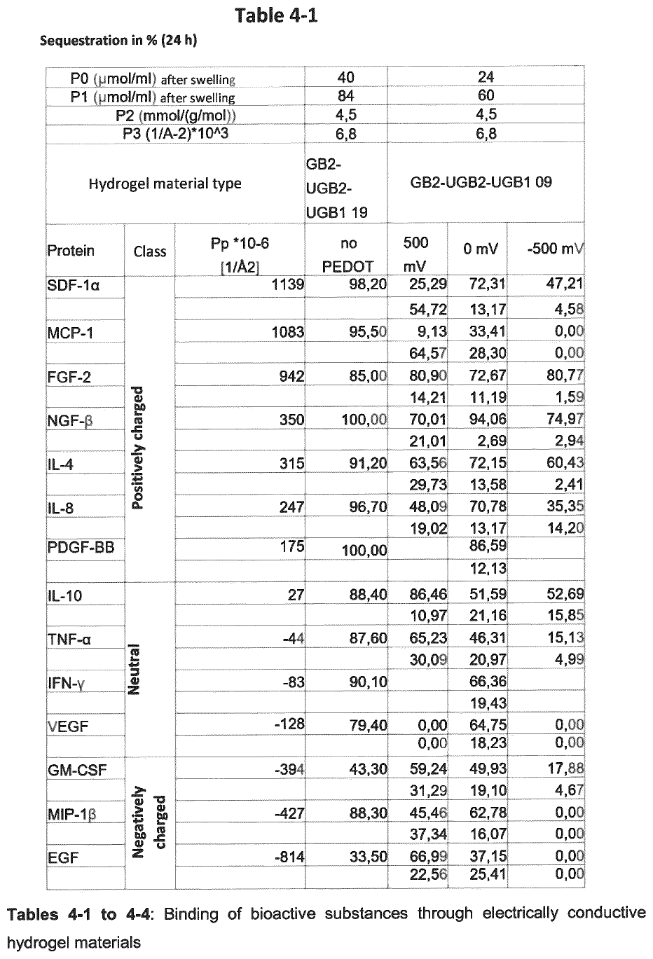

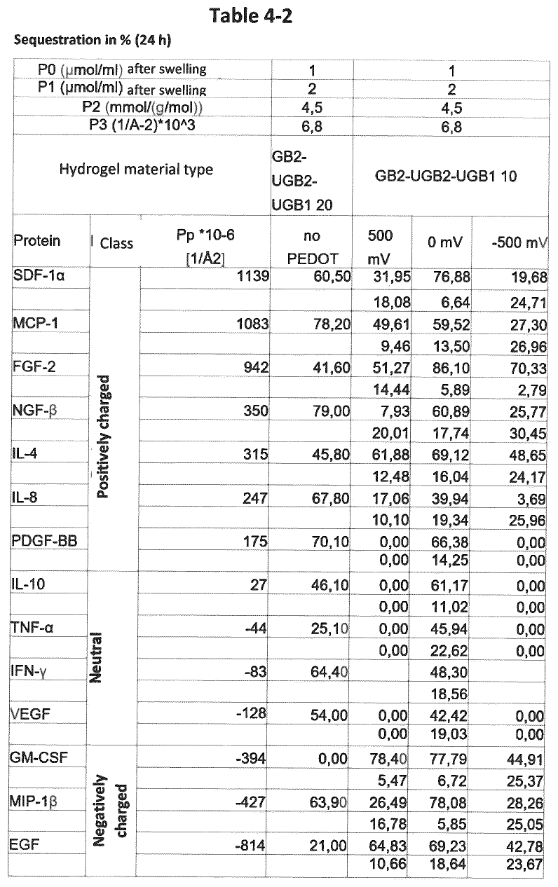

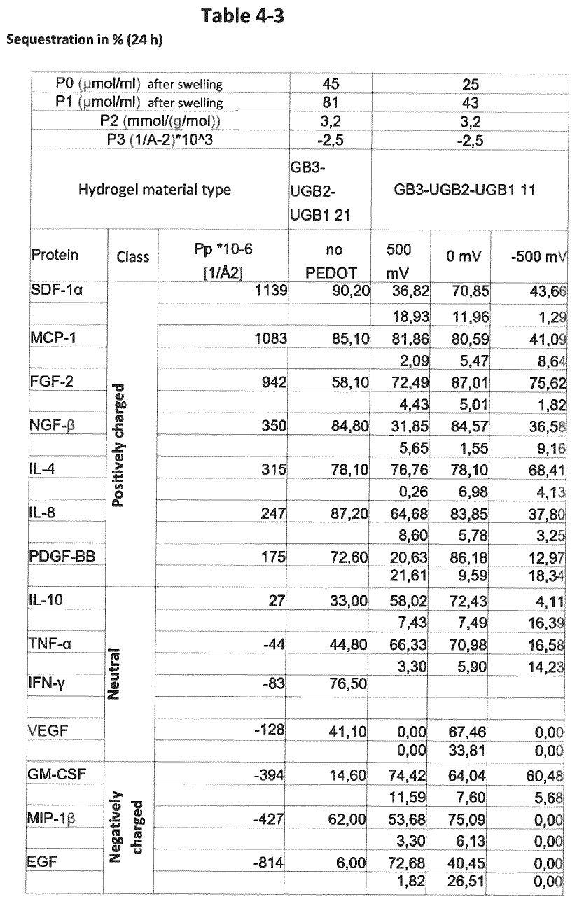

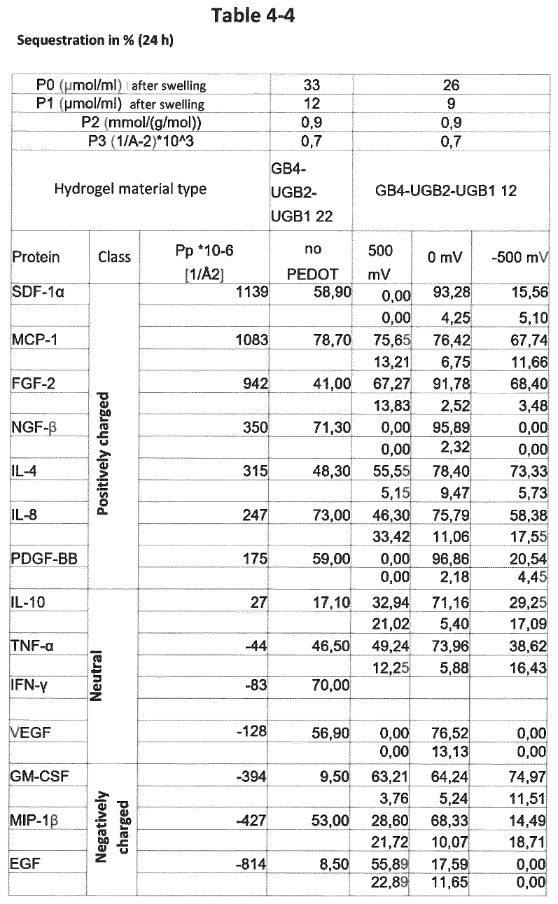

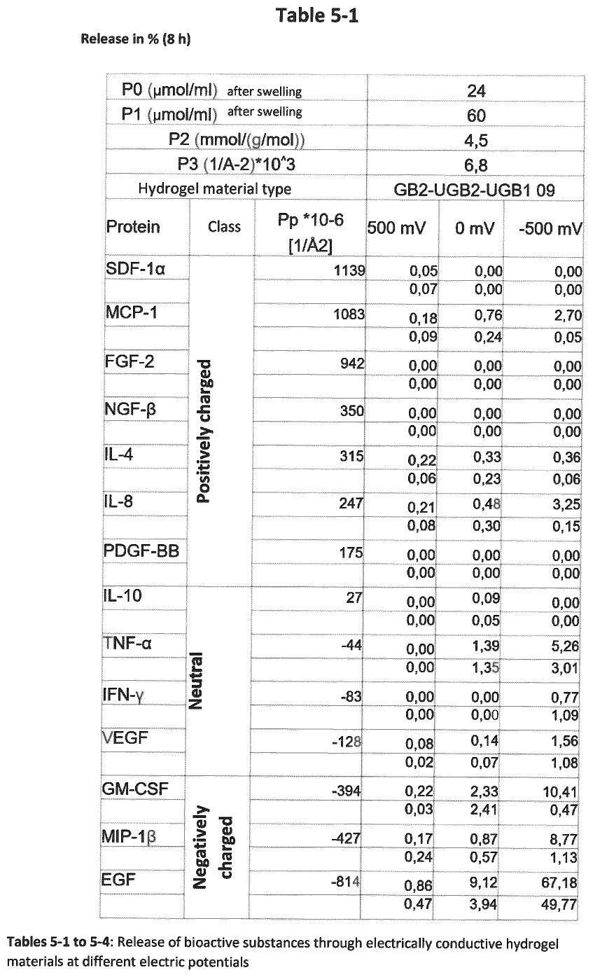

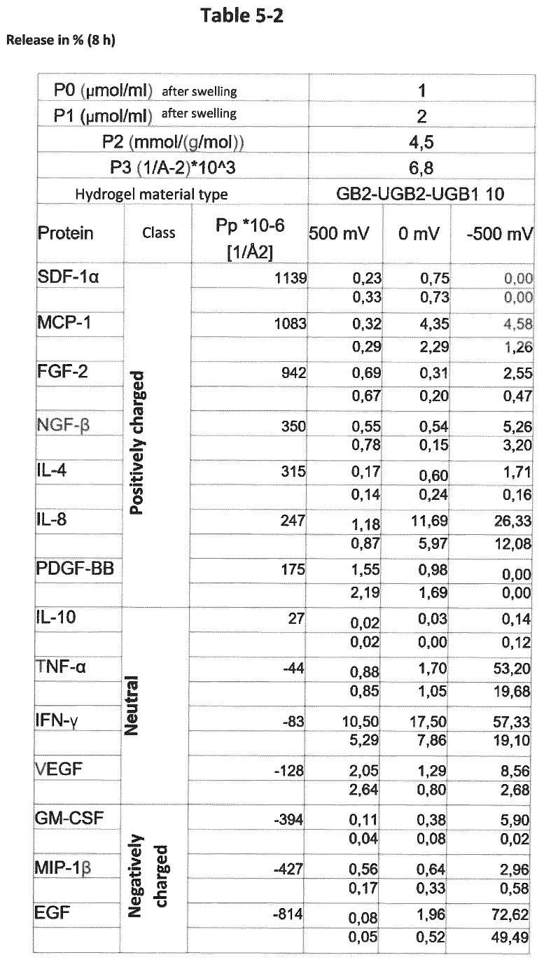

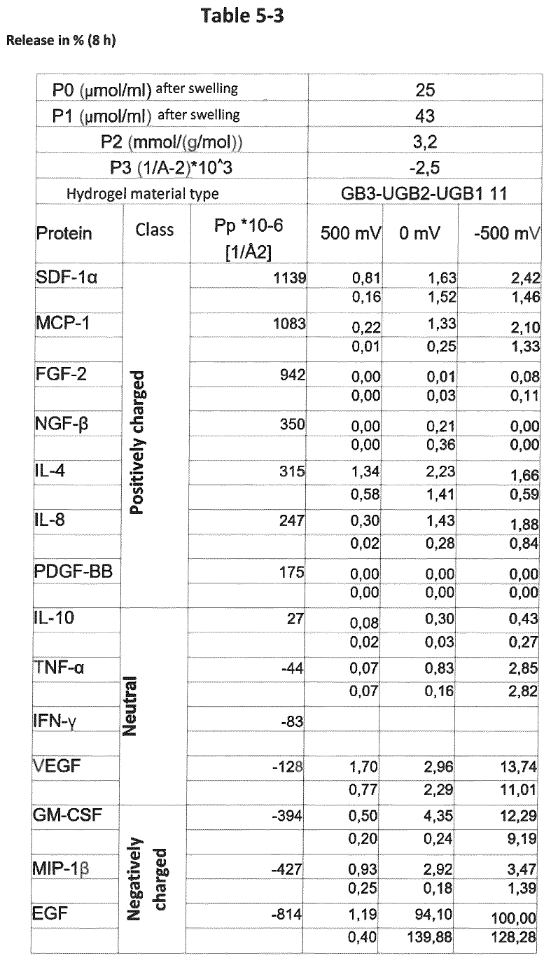

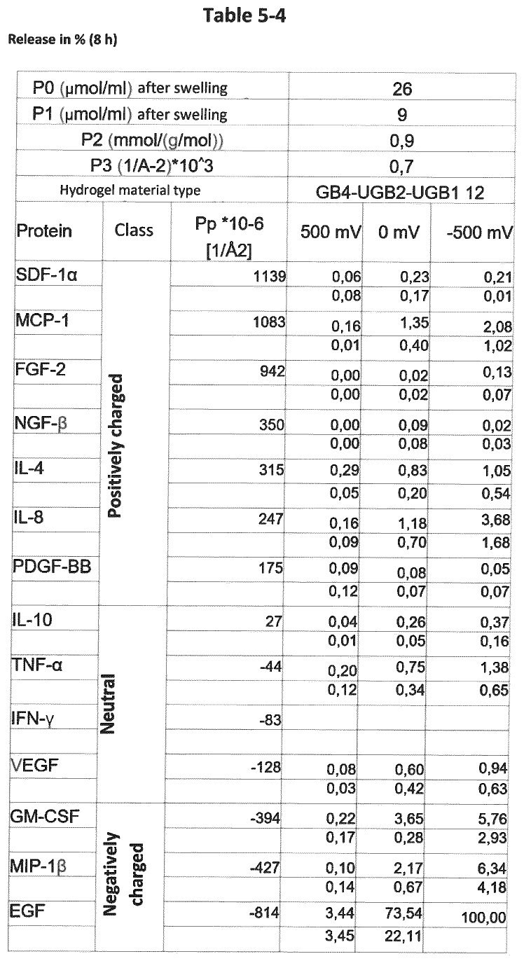

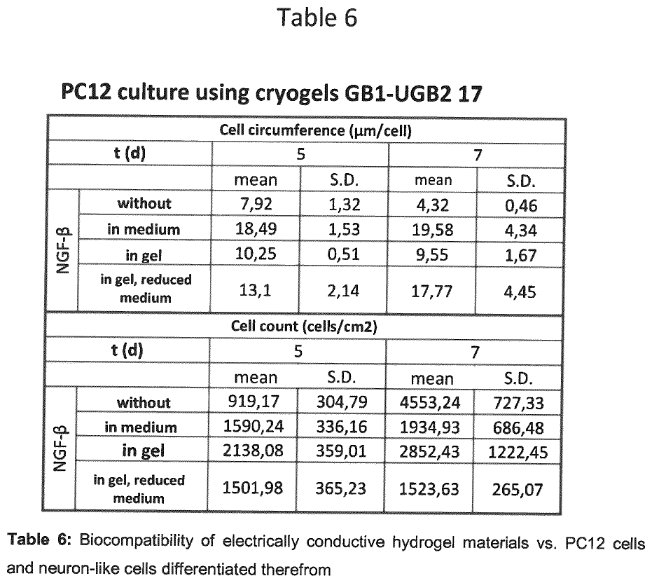

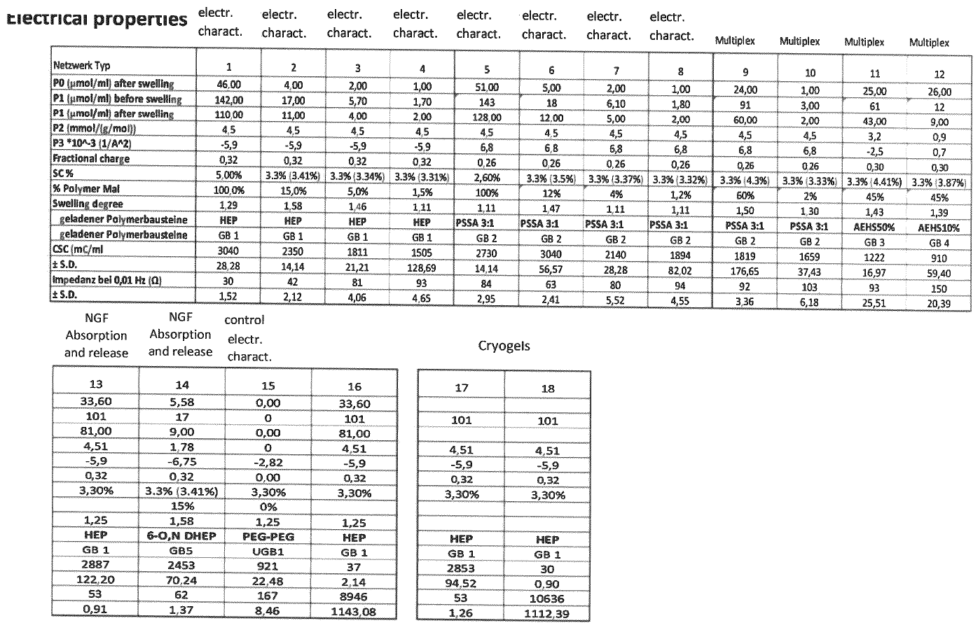

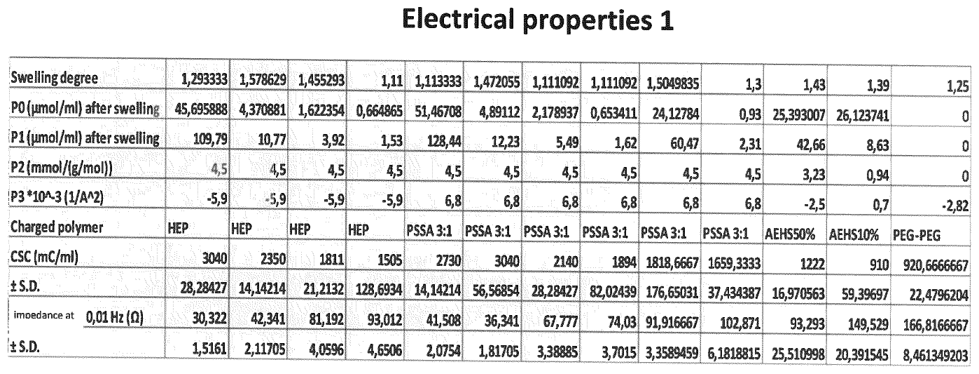

FIG. 3: an image showing the cross-sectional visual appearance of the electrically conductive porous and non-porous hydrogel materials;

Anionically charged hydrogel building blocks are abbreviated as GB in the following, wherein different anionically charged building blocks are additionally identified by a reference numeral. Uncharged building blocks of the hydrogel material are abbreviated as UGB, wherein different uncharged building blocks are identified by a reference numeral. For the sake of simplicity and for brevity, hydrogel materials are referred to in the tables as hydrogels. Hydrogel material types are referred to as hydrogel types in the tables.

FIG. 1 shows a schematic representation for explanation of the electrically conductive hydrogel material 1 according to the invention. Illustration A of FIG. 1 shows by way of example the synthesis of the polymer network 2 from anionically charged building blocks 3 and crosslinker molecules 4 as a template for forming the hydrogel material 1 according to the invention. The parameter configuration with the parameters P1, P2 and P3 is predetermined with the polymer network 2. Illustration B shows the structure of the electrically conductive hydrogel material 1 as a pseudo-Interpenetrating polymer network (IPN) of the polymer network 2 and an electrically conductive component 5. The polymer network 2 carries anionic groups and is determined by the parameters P1, P2 and P3. The electrically conductive hydrogel material 1 is formed by polymerization or crosslinking of the polymer network 2 with the electrically conductive component 5. In illustration C of FIG. 1, the doping of the conductive component 5 is shown by way of example. In the example, the electrically conductive component 5 is PEDOT: The doping takes place via the interaction with sulfate/sulfonate groups in the anionically charged polymer network 2. The integral charge density P1 of the polymer network 2, the local charge density P2 and the hydrophobicity P3 of the polymer carrying anionic groups in the polymer network 2 are decisive for the interaction with PEDOT 5 and the resulting electrical properties of the conductive hydrogel material 1. Illustration D represents by way of example the influence of the application of an electric potential on the electrically conductive hydrogel material. The positive charge of the PEDOT can be continuously regulated by the influence of an electrical potential. The regulation takes place from neutral (0) via medium (+1) to strongly (+3) positively charged.

FIG. 2 shows a further schematic representation for further explanation of the invention. An electrically conductive hydrogel material 1 is represented which is formed from sulfated and sulfonated polymers as charged building blocks 3 and PEG as crosslinking agents 4 which form a polymer network 2, and PEDOT as electrically conductive component 5 which is incorporated in the polymer network. On the right with the reference numeral 6, a positively charged signal protein is represented which is bound to negatively charged PEDOT. The binding properties of the signal protein 6 in the hydrogel material 1 can be influenced by the influence of an electrical potential. For electrically influencing the hydrogel material 1, the hydrogel material 1 is electrically contacted to a voltage source 7. The hydrogel material 1 according to the invention allows an electrodynamic modulation of specific electrostatic interactions between the hydrogel polymer and the signal protein 6.

### EXAMPLE EMBODIMENTS

### Synthesis of the Polymer Network

For the synthesis of the hydrogel material 1, three anionically charged polymer network systems 2, (1) one consisting of two hydrogel building blocks, an anionically charged building block (GB1-5) with maleimide groups and an uncharged building block (UGB2) with thiol groups (hereinafter referred to as maleimide thiol two component system) or (2) one consisting of three hydrogel building blocks (an anionically charged building block (GB1-5) with maleimide groups and an uncharged building block with maleimide groups (UGB1)) and an uncharged building block (UGB2) with thiol groups (hereinafter referred to as maleimide thiol three component system) or (3) a system consisting of two hydrogel building blocks crosslinked via EDC/NHS-based activation of the carboxyl groups of the anionically charged hydrogel building block (GB1) and reaction with the amino groups on the second, uncharged hydrogel building block (UGB3) (hereinafter referred to as EDC/NHS system) were used.

The properties of the anionically charged hydrogel building blocks (GB1 to GB5) and of the uncharged hydrogel building blocks (UGB1 to UGB3) can be taken from Table 1.

### Maleimide-Thiol Two-Component System for Producing an Anionically Charged Polymer Network as a Precursor of the Hydrogel Material According to the Invention

GB1 (heparin maleimide, 15 kDa) and UGB2 (star-shaped thiol-functionalized polyethylene glycol, starPEG, 10 kDa) are dissolved in 0.1× phosphate-buffered saline (PBS), pH=6) in a concentration of 0.0015 mol/l each. 1×PBS consists of 137 nm NaCl, 2.7 mM KCl and 12 mM total phosphate consisting of HPO₄²⁻ and H₂PO₄. The mixing ratios and concentrations can be taken from Table 2. All subsequent steps until the hydrogel building blocks are mixed are carried out on ice. (pH value adjustment for achieving a gelling time of 30 min) For a molar ratio of 1 of the two building blocks, identical volumes of the two solutions are mixed by mixing by means of a pipette and/or in a mixing device. The mixture is then centrifuged in order to remove air bubbles and the samples are pipetted onto gold electrodes (diameter 11 mm, 100 μl) or onto a cover glass (diameter 8 mm, 67 μl) and covered with a hydrophobic (Sigmacote® treated) cover glass 11 mm or 8 mm in size. The crosslinking of the hydrogel is effected by the reaction of the thiol and maleimide groups (Michael addition reaction). The polymerization is carried out for at least 30 min at room temperature in a moist chamber in order to avoid drying out of the gels. For the cryogelling, the samples are polymerized overnight at −15° C. The completely polymerized hydrogels are then swollen overnight in 1×PBS (0.154 mmol/l NaCl, pH 7.4). The solids content of the gels is about 3% (m/v).

### Maleimide-Thiol Three-Component System for Producing a Polymer Network as a Precursor of the Hydrogel Material According to the Invention

A three-component system is used for precise adjustment of the integral negative charge in the hydrogel material. StarPEG thiol, starPEG maleimides and maleimide-functionalized anionically charged hydrogel building blocks polymers are dissolved in 0.01×PBS in a predetermined concentration and mixed in various ratios as a function of the desired integral charge. The mixing ratios can be taken from Table 2. By adding 0.01 M HCl or 0.01 M NaOH, the time for gelling is adjusted to less than 5 min. The solutions are then mixed and the sample body is produced at room temperature (30 min) or 15° C. (overnight) in a manner analogous to the two-component system. The fully polymerized hydrogels are swollen overnight in 1×PBS (pH 7). The solids content of the gels is about 3% (m/v).

The properties of the uncharged and anionically charged building blocks are shown in Table 1.

### EDC/NHS System for Producing a Polymer Network as a Precursor of the Hydrogel Material According to the Invention

For the EDC/NHS system, 0.167 mg/μl of UGB3, 0.145 mg/μl of GB1, 0.1 mg/μl of EDC (N-(3-dimethylaminopropyl)-N′-ethylcarbodiimide) and 0.1 mg/μl of NHS (N-hydroxysulfosuccinimide) are dissolved in ice-cold milliQ water for a molar ratio of UGB3 stemPEG amine to non-maleimized GB1 of 2. For 1 ml of final gel volume, first 85.20 μl EDC solution and 48.25 μl NHS solution are pipetted to 533.22 μl heparin solution, mixed in a laboratory mixer (vortexing) and incubated on ice for 15 min. 333.33 μl of starPEG solution are then added and the mixing is repeated using vortexes. Directly thereafter, the desired shaped bodies are produced identically to the two-component system. The polymerization is carried out over night at room temperature or −15° C. in a moist chamber in order to avoid drying out of the hydrogels. The total solids content of the gels is approximately 10% at a final starPEG concentration of 0.00784 mol/l and a final heparin concentration of 0.00392 mol/l. The fully polymerized hydrogels were swollen overnight in 1×PBS (pH 7).

### Synthesis of the Electrically Conductive Hydrogel Material as a Pseudo-Interpenetrating Network

The synthesis of the electrically conductive pseudo-interpenetrating polymer network is possible for all hydrogel materials of the above-described formation rules. For the synthesis of the electrically conductive pseudo-interpenetrating poly-3,4-ethylenedioxythiophene (PEDOT) network with PEDOT as the electrically conductive component, the completely swollen anionically charged polymer networks are incubated in the first step for 3 hours in a 0.4 M ammonium peroxodisulfate (APS) solution in 1 M HCl at room temperature. The hydrogel is then incubated for 6 hours in a 0.4 M 3,4-ethylenedioxythiophene (EDOT) solution in mineral oil in a rotary shaker. During this time, the oxidative polymerization of the EDOT takes place to form an electrically conductive PEDOT network which assumes a black color with increasing polymerization of the PEDOT (FIG. 3). The hydrogel materials thus produced are then washed overnight in mineral oil in order to remove unreacted monomers. In order to remove the mineral oil, the hydrogel materials are then washed in hexane. The washing process can be repeated. The hydrogel materials are then washed for at least 24 h in PBS (pH 7.4).

All hydrogel material types are summarized in Table 2. The concentrations given are the concentration in the finished gel mixture. The hydrogel material types GB1-UGB2 16, GB1-UGB2 18, GB2-UGB2-UGB1 19, GB2-UGB2-UGB1 20, GB3-UGB2-UGB1 21 and GB4-UGB2-UGB1 22 are hydrogels which have not been functionalized with PEDOT. Hydrogel material type UGB1-UGB2 15 is a pure PEG-PEG hydrogel which does not carry any anionic charges, but PEDOT is functionalized.

### Electrical Characterization

The electrical characterization of all hydrogels was carried out by means of impedance spectroscopy or cyclic voltammetry in a 3-electrode setup in 100 ml of 1×PBS (pH 7). For both measuring methods, 100 μl gel sample bodies (unswollen volume) were produced on a gold net electrode which serves as a working electrode. A porous carbon electrode (BioLogic A-010530) with a significantly larger surface area compared to the working electrode was used as the counter electrode. An Ag/AgCl electrode (Metrohm, Part No. 6.0726.100) served as the reference electrode. A potentiostat (Metrohm Autolab PGSTAT204 or PalmSens 4) served as the measuring device.

### Impedance Spectroscopy

For impedance spectroscopy, an effective potential of 10 mVₘₛ is applied and the impedance and the phase angle are measured in a frequency range from 0.01 Hz to 10⁵ Hz at 10 measurement points per decade. The impedance of the various hydrogel material types was compared at a frequency of 0.01 Hz. The electrical conductivity is inversely proportional to the impedance. If the impedance increases, the electrical conductivity decreases.

### Cyclic Voltammetry

The charge storage capacity is calculated by measuring the cyclic voltammetry. For this purpose, the current flow between the working electrode (hydrogel) and the counter electrode (carbon electrode) is measured using the above-mentioned 3-electrode structure, while the applied electrical potential of −0.6 to 0.8 V (potential between working electrode and Ag/AgCl reference electrode) is varied in 5 cyclic passes. The scan rate is 50 mV/s. Subsequently, the negative part of the area under the curve is integrated (MultiTrace 4.3, PalmSens 4) and the charge storage capacity of the hydrogel is calculated from this with the following formula:

$$charge storage capacity = \begin{matrix}I * U \\ scan rate\end{matrix}$$

As an integral, the software outputs the value I*U in the unit [A]*[V]. [A]=[C/s] applies. The division of the scan rate [V/s] results in the charge [C] which is transmitted by the material. This is then set in the ratio to the volume, since the interface to the surrounding medium is present in the complete volume of the hydrogel and thus no surface can be calculated. The values are expressed in charge per milliliter of hydrogel.

### Electrical Properties of the Hydrogel Materials

Immediately after oxidative polymerization, PEDOT possesses an inherent positive charge (10.1021/acs.jpcb.9b01745, 10.1021/acsapm.8b00061). Due to the non-covalent interactions between PEDOT and the anionically charged polymer network (hydrogel) and the non-covalent interaction between the individual PEDOT polymer chains, a pseudo-interpenetrating network is formed between the anionically charged polymer network and PEDOT. At the same time, the negative charges of the anionically charged polymer network function as a p-doping for PEDOT. Depending on the integral and local charge density (parameters P0/P1 and P2), the doping degree of the PEDOT changes. This has a direct influence on the electrical properties (impedance and charge storage capacity) of the electrically conductive hydrogel material. At the same P2 and P3, a charge storage capacity of 3040 to 1505 mC/ml and an impedance of 30 to 93Ω for the hydrogel material types GB1-UGB2 01 and GB1-UGB2-UGB1 02-04 over a P1 range from 110 to 2 2 μmol/ml and a charge storage capacity of 3040 to 1894 mC/ml and an impedance of 36 to 74Ω for the hydrogel material types GB2-UGB2 05 and GB2-UGB2-UGB1 06-08 in a P1 range from 128 to 2 μmol/ml were obtained (Table 3-1 and 3-2). Since PEDOT is a hydrophobic polymer, the distribution and crosslinking of the PEDOT chains among themselves is strongly dependent on the hydrophobicity of the environment and thus dependent on the amphiphilia of the anionically charged polymer network (parameter P3), which also has an influence on the electrical properties of the electrically conductive hydrogel material. In the exemplary embodiments, it is shown that with a high value of the parameter P3 of 6.8*10⁻³ 1/A² (GB2-UGB2-UGB1 08), see Table 2 In comparison to a low value for P3 of −5.9*10⁻³ 1/A² (GB1-UGB2-UGB1 04), with the value remaining the same for P2 (4.5 mmol/(g/mol)) and P1 (2 μmol/ml), the higher hydrophobicity leads to a higher charge storage capacity (1894 vs. 1505 mC/ml) and a lower impedance (74Ω vs. 93Ω) (Table 3-1 and 3-2). If the distance between the strongly anionically charged groups is increased along the polymer chains (reduced value P2, stands for a lower local charge density), a significantly lower charge storage capacity with increased impedance is obtained with similar P3 and approximately the same P1. This difference is particularly evident in a comparison with low P1 of 9 mmol/ml, P2 of 0.9 mmol/(g/mol) and P3 of 0.7*10⁻³ 1/A² (GB4-UGB2-UGB1 12) in comparison with a P1 of 11 or 4 mmol/ml, P2 of 4.5 mmol/(g/mol) and P3 of −5.9*10⁻³ 1/A² (GB1-UGB2-UGB1 02/03). Despite the lower value for P3 and an approximately equal or lower value for P1, the hydrogels with higher P2 (GB1-UGB2-UGB1 02/03) have a significantly higher charge storage capacity (2350 and 1811 mC/mi and lower impedance (42 and 81Ω) compared to GB4-UGB2-UGB1 12 (910 mC/ml and 150Ω). Despite the moderate values for P1 and P3, GB4-UGB2-UGB1 12 behaves like a hydrogel without anionically charged groups after PEDOT functionalization, see UGB1-UGB2 15 (pure PEG hydrogel material, 921 mC/ml and 167Ω). Conductive hydrogels having different electrical properties can thus be obtained by the changes in the doping degree (P1, P2) and the hydrophobicity (P3). By means of a high integral number of anionic groups (i.e. a high value for P0 or P1) and a small distance between the strongly anionically charged groups (P2), hydrogels having a high conductivity can be obtained. P0 appears to have a similar influence on the electrical properties as P1, which is probably due to the proportion of the strongly anionic groups. The weakly anionic groups, in this case, for example, carboxyl groups having an intrinsic pKa in the range from 3.5 to 4.5, probably play a minor role in the doping of the PEDOT. The interaction between the hydrophilic/amphiphilic anionic polymer and the hydrophobic electrically conductive polymer (PEDOT) can likewise be configured by parameter P3. Increased hydrophobicity of the anionically charged hydrogel building blocks (high value for parameter P3) allows increased conductivity (i.e. lower impedances) to be generated even at a significantly lower P1. This is due to the high affinity of the hydrophobic PEDOT units to the hydrophobic groups on the anionically charged building block, which has a positive influence on the doping. In addition, the hydrophobic groups already facilitate the penetration and distribution of the monomer units (EDOT) in the anionically charged polymer network during the PEDOT synthesis. The electrically conductive hydrogel materials with very high conductivity (low impedance) and high charge storage capacity can be used as biocompatible electrode materials for stimulation of cells or tissues. Due to the high hydration and softness of these hydrogels, which are very similar to biological tissue, the foreign body reactions of the organism can be significantly reduced in comparison with those electrodes based on the electrically conductive hydrogel materials according to the invention in comparison with classical metal electrodes. In addition, in classical metal electrodes, the charge transfer for the electrical stimulation takes place only at the contact surface from the metal to the tissue (physiological solution). Due to the distribution of the conductive polymer within the volume material and the possibility that biofluids can diffuse into the hydrogel, a charge transfer can take place within the complete volume. As a result, lower voltages can be used for the same charge injection, which reduces the development of heat and potential tissue damage resulting therefrom in comparison with conventional metal electrodes.

### Active Substance Sequestration and Release

The sample preparation for the active substance sequestration and release takes place identically to the characterization of the electrical properties. 100 μl of the various anionically charged polymer networks were applied to a gold network electrode and made electrically conductive by means of subsequent penetration and polymerization of EDOT monomers to PEDOT.

### Active Substance Sequestration

After thorough washing of the electrically conductive hydrogel materials in PBS, the sequestration of various substances with a concentration of in each case 100 ng/ml of substance in 2.5 ml of PBS was carried out with 0.1% BSA (in order to simulate the physiological situation with a carrier protein). This corresponds to 250 ng per protein and hydrogel material. Sequestration is carried out in a 5 ml low binding Eppendorf tube (Eppendorf) in order to minimize non-specific binding of the proteins to the reaction vessel. The absorption takes place over 24 h. Sequestration is carried out at 500 mV, 0 mV (passive) and −500 mV. The active sequestration takes place in a 3-electrode setup similar to the electrical characterization. The conductive hydrogel on the gold electrode served as the working electrode. This was located together with the reference electrode, an Ag/AgCl wire, in a 5 ml low-binding Eppendorf tube. The counter electrode, a porous carbon electrode (BioLogic A-010530) with a significantly larger surface area compared to the working electrode, was located in a separate vessel with 100 ml of PBS. The circuit was closed by a salt bridge consisting of a PVC hose which was filled with a 25% polyacrylamide hydrogel (indicated by the manufacturer) swollen in PBS. In the Eppendorf vessel, the tube was additionally closed with a 1000 Da dialysis membrane to prevent penetration of the substances. A potentiostat (Metrohm Autolab PGSTAT204 or PalmSens 4) was used to create a defined potential. 100 μl of the solution were taken as samples before and after the sequestration. After determining the concentrations of the various proteins according to the manufacturer's instructions using the multiplex assay kit (Luminex Technology, ThermoFisher), the amount of protein absorbed was calculated as a percentage.

For the sequestration of substances, the integral P0 or P1 and local charge density (P2) at the various applied voltages in the entire hydrogel material (anionically charged polymer network-PEDOT pseudo IPN) play a predominant role. The highest sequestration could be measured independently of the charge of the bound molecule without applied potential (Table 4-1, 4-2, 4-3 and 4-4). In comparison with non-conductive hydrogels (without PEDOT) with similar P1 and P2, it is shown that the electrically conductive hydrogel materials have a lower absorption of positively charged substances (GB2-UGB2-UGB1 09; 72.3% SDF-1α; 72.7% FGF-2; 70.8% IL-8 over GB2-UGB2-UGB1 19; 98.2% SDF-1α; 85.0% FGF-2; 96.7% IL-8) (see Table 4-1, 4-2, 4-3 and 4-4). Negatively charged molecules have a reverse effect. An uptake of 49.9% GM-CSF and 37.1% EGF for GB2-UGB2-UGB1 09 compared to 43.3% GM-CSF and 33.5% EGF for GB2-UGB2-UGB1 19 could be measured. This effect is even more pronounced with a lower P1. Thus, 77.8% GM-CSF and 69.2% EGF were bound for GB2-UGB2-UGB1 10 compared to 0.0% GM-CSF and 21.0% EGF for GB2-UGB2-UGB1 20. The reason for this is probably the positive charge of the PEDOT. Without applying an electrical potential, PEDOT has a positive charge of about 33% of the monomer units which can cause an interaction with negatively charged substances and can probably bind them (10.1021/acsapm.8b00061, 10.1021/acsami.5b04768). In addition, it has been shown that with a low P1, positively charged substances could also be bound better by the electrically conductive hydrogels in comparison to the non-conductive control. For example, for GB2-UGB2-UGB1 10, 76.9% SDF-1α; 86.1% FGF-2; 39.9% IL-8 could be bound compared to GB2-UGB2-UGB1 20, 60.5% SDF-1α; 41.6% FGF-2; 17.1% IL-8. This suggests that the conductive polymer PEDOT plays an important role for the binding of predominantly positively charged substances, in particular by mutual charge compensation with the anionically charged groups of the hydrogel material. Both the ionic binding of negatively charged domains in the protein by PEDOT and hydrophobic interactions between protein and the PEDOT play an important role here.

If a positive potential of 500 mV is applied, the overall sequestration of substances is reduced (Table 4-1, 4-2, 4-3 and 4-4). This can be observed above all in the case of positively charged substances. In the case of negatively charged substances, such as, for example, GM-CSF and EGF, on the other hand, a slight increase or a nearly identical sequestration can be observed. Thus, 49.9% GM-CSF and 37.1% EGF before applying the potential and 59.2% GM-CSF and 67.0% EGF after applying the potential at GB2-UGB2-UGB1 09 respectively 77.8% GM-CSF and 69.2% EGF before applying the potential and 78.4% GM-CSF and 64.8% EGF after applying the potential at GB2-UGB2-UGB1 09 could be bound. The reason for this may be that the charge of the PEDOT increases by applying a positive potential. (10.1021/acsapm.8b00061, 10.1021/acsami.5b04768). This leads to a positive influence on the binding of negatively charged proteins such as GM-CSF and EGF by the increased ionic interaction. Since, on the other hand, the negative charge of the anionic component in the hydrogel is constant and the stronger positive charge of the PEDOT can lead to partial charge compensation, the absorption of positively charged proteins is reduced summarily.

If a negative potential is applied, the positive charge of the PEDOT can be reduced or even completely neutralized (10.1021/acsapm.8b00061, 10.1021/acsami.5b04768). At a potential of −500 mV, a reduction of the substance sequestration is shown predominantly independently of the charge of the substances and independently of the P1 and P2 of the hydrogel materials (Table 5-1, 5-2, 5-3 and 5-4). Thus, the sequestration of SDF-1α decreases from 72.3% to 47.2%, NGF-β from 94.1% to 75.0% and IL-8 from 70.8% to 35.4% in GB2-UGB2-UGB1 09. A similar trend can be observed for GB2-UGB2-UGB1 10 and GB3-UGB2-UGB1 11/12. This confirms the assumption that the positive charge of the PEDOT plays an important role for the binding of predominantly positively charged substances. This can be caused, e.g., by the binding of negatively charged domains of the substances (proteins) to the positive charges of PEDOT, the phenomenon described above. In the sequestration of negatively charged substances, a very strong reduction in the sequestration is shown at −500 mV. Thus, the sequestration of GM-CSF decreases from 49.9% to 17.9% and of EGF from 37.1% to 0.0% in GB2-UGB2-UGB1 09 and of GM-CSF from 77.8% to 44.9% and EGF from 69.2% to 42.8% in GB2-UGB2-UGB1 10. The reason for this strong reduction of the sequestration of negatively charged substances at negative potential is probably the reduction of the positive charge of the PEDOT. As a result, significantly fewer binding sites are found for negatively charged substances.

### Active Substance Release

After the sequestration (loading of the hydrogels according to the third loading method mentioned above), the electrically controlled release of the bioactive molecules took place. On the basis of the highest average sequestration, the samples were used for this, which were loaded with substances at a potential of 0 mV (passive) for 24 h. A controlled release of actively sequestered substances can likewise be carried out according to the protocol described below.

After loading the hydrogels, a short washing step was initially carried out to remove weakly bound proteins. For this purpose, the hydrogel is first centrifuged at 3000 rpm for 1 min in order to remove adhering liquid. The hydrogels are then washed with BSA in 1 ml of PBS and centrifuged again at 3000 rpm for 1 min. The release is likewise carried out in the 3-electrode setup described in the chapter on active substance sequestration at an applied potential of 500 mV, 0 mV and −500 mV. The sample was taken for the starting solution (control), after sequestration, after washing and for release after 0 min, 10 min, 30 min, 1 h, 6 h, 8 h and 24 h. Since saturation of the released proteins could be observed after 8 hours, the results of the plateau release are shown after 8 hours (Table 5-1, 5-2, 5-3 and 5-4). The concentration of the various substances was determined according to the manufacturer's instructions using the multiplex assay kit (Luminex Technology, ThermoFisher).

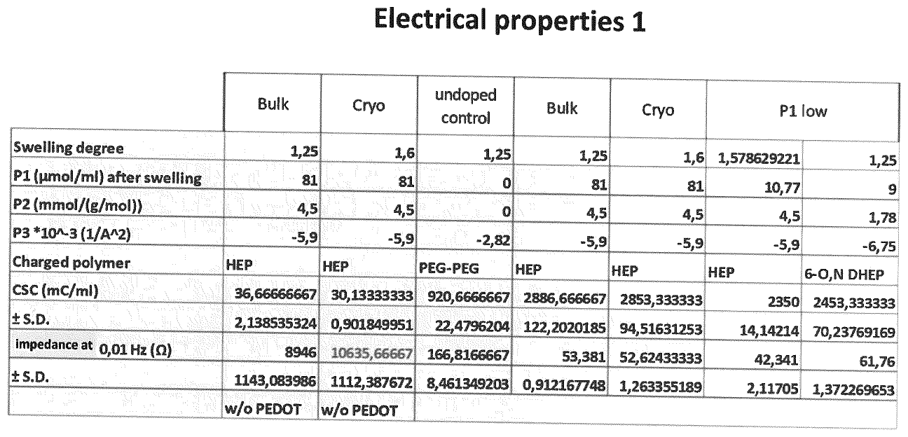

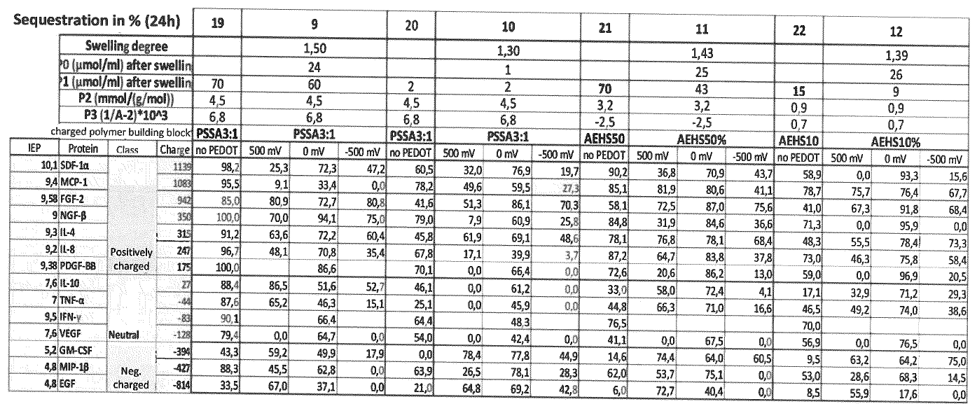

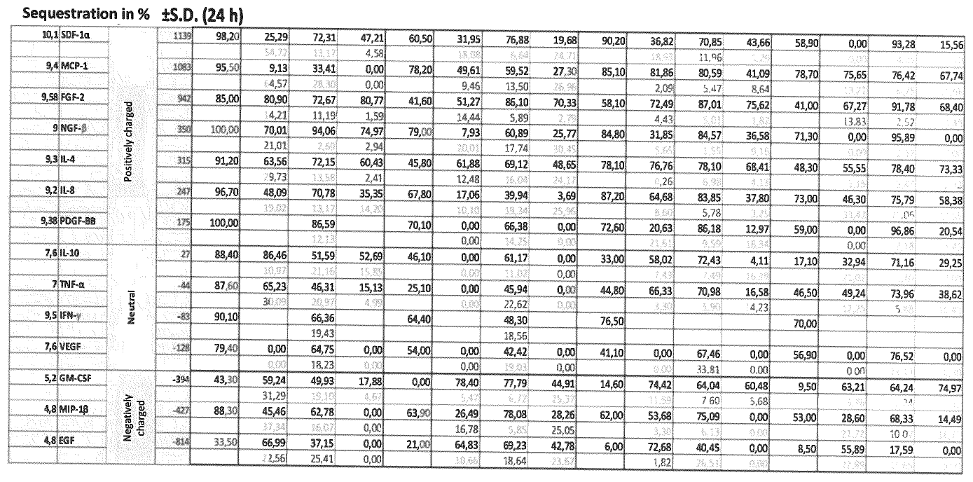

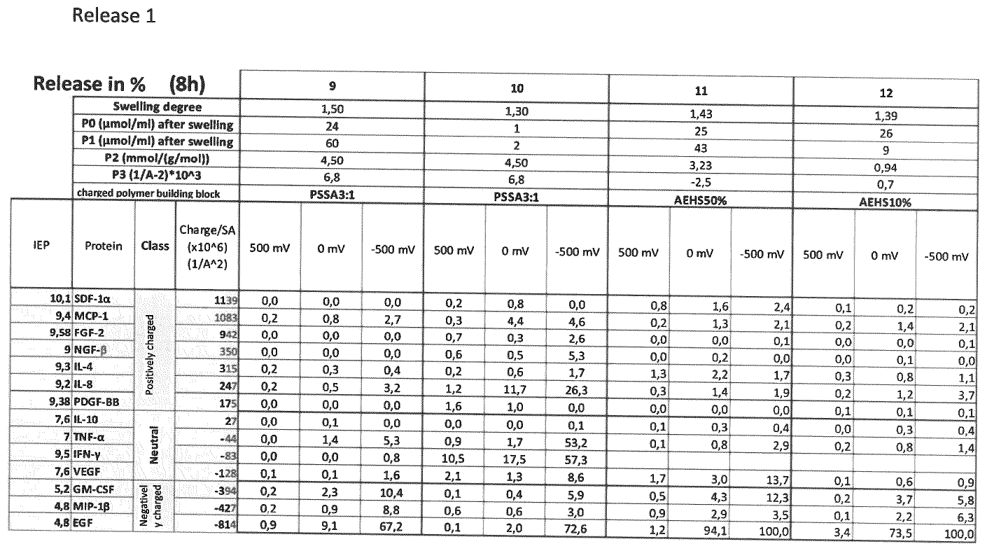

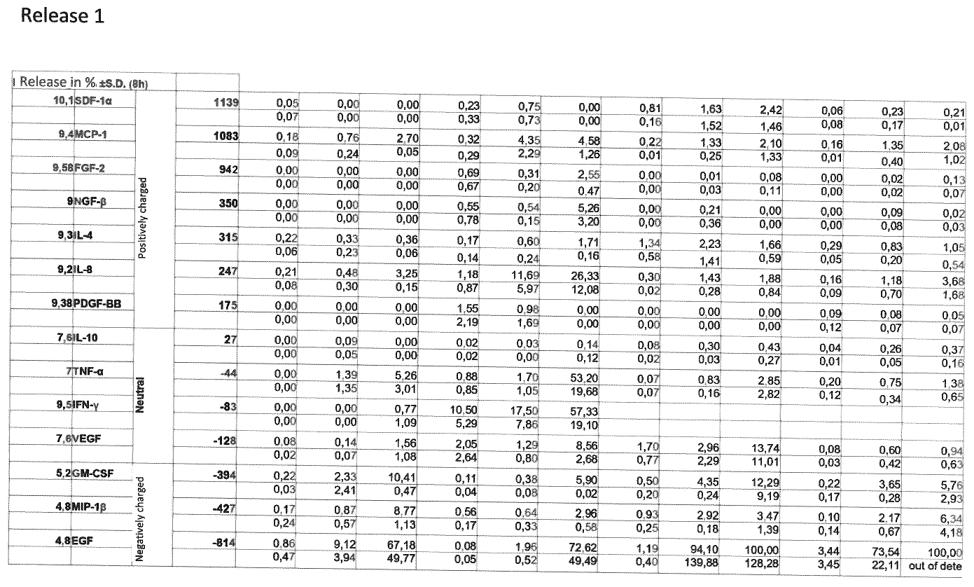

The release of the bound substances can be adjusted as a function of the configured parameters of the anionically charged polymer network in combination with the charge of the PEDOT (Table 5-1, 5-2, 5-3 and 5-4). At a high P1 (60 μmol/ml, GB2-UGB2-UGB1 09) only a very low release of positively charged substances (0.0% SDF-1α; 0.0% FGF-2; 0.5% IL-8) takes place. For positively charged substances, however, a moderately low release can be observed (2.3% GM-CSF; 9.1% EGF). Due to the probable overcompensation of the positive charge of the PEDOT by the anionic groups, a release of negatively charged substances can probably take place significantly faster than the release of positively charged proteins. At a low P1 (2 μmol/ml, GB2-UGB2-UGB1 10) a very increased release of the positively charged substances (0.8% SDF-1α; 0.3% FGF-2; 11.7% IL-8) takes place. Negatively charged substances, on the other hand, are released less compared with a higher P1 (0.4% GM-CSF; 2.0% EGF). If P2 is reduced at similar P1 values (GB3-UGB2-UGB1 11/12), an increased release of negatively charged substances (4.3/3.7% GM-CSF; 94.1/73.5% EGF) with virtually unchanged release of positively charged substances (1.6/0.2% SDF-1α; 0.0/0.0% FGF-2; 1.4/1.2% IL-8) can be achieved (FIG. 8, Table 5). The reason for this is also the compensation of positive charges in the PEDOT by anionic groups despite the lower charge density.

If a potential of 500 mV is applied, a retention of bound substances can be observed independently of their charges. The effect is particularly clear for GB2-UGB2-UGB1 09. Here, the release of nearly all 14 proteins could be reduced to 0% (Table 5-1, 5-2, 5-3 and 5-4). Even with moderate P1 (9 μmol/ml) and very low P2 (0.94 mmol/(g/mol)) in GB3-UGB2-UGB1 12, the release of all substances could be reduced to almost 0% (Table 5-1, 5-2, 5-3 and 5-4). The reason for this could be the swelling rate of the hydrogels. At a positive potential, the increased positive charge of the PEDOT compensates for more anionically charged groups. Assuming that more anionic groups are present in the hydrogel material compared to the positive charges of the PEDOT, the entire charge of the hydrogel shifts toward the neutral, as a result of which a loss of water of the hydrogel occurs and thus shrinkage occurs. This leads to a reduced mesh size of the polymer chains, which in turn results in a reduced release of the proteins independently of their charge.

If a potential of −500 mV is applied, an increased release is shown for a large part of the substances (Table 5-1, 5-2, 5-3 and 5-4). This relates predominantly to negatively charged bioactive molecules. Thus, after the potential has been applied, the release of TNF-α, GM-CSF and EGF for the hydrogel material type GB2-UGB2-UGB1 09 increases from 1.4/2.3/9.2% to 5.3/10.4/67.2% and GB2-UGB2-UGB1 10 from 1.7/0.4/2.0% to 53.2/5.9/72.6%. This trend can also be observed for the hydrogel material types GB3-UGB2-UGB1 11/12. It is interesting here that substances which already have a greater release without applied potential are released most by applying the negative potential. The reason for this is most likely the reduction/neutralization of the positive charge of the PEDOT. As a result, there are more free anionic groups which cause rejection of negatively charged proteins by ionic interactions. However, an increased release can also be observed with positively charged substances. Thus, the release of FGF-2, IL4 and IL8 for the hydrogel material type GB2-UGB2-UGB1 10 increases from 0.3% to 2.6%, 0.6% to 1.7% and 11.7% to 26.3%. The reason for this could likewise be the increased number of free anionically charged groups due to the lack of charge compensation with PEDOT. The resulting increased negative net charge of the hydrogel leads to a swelling. This promotes release independently of the charge, as a result of which positively charged substances can also be released to a greater extent.

### Hydrogel Biocompatibility

The PC12 cell line and the hydrogel material type GB1-UGB2 17 were used for the investigation of biocompatibility. The cell line is a phaeochromocytoma cell which can be differentiated into neuron-like cells within a few days by adding NGF-β. The PC-12 cell line is a widely used model for neural differentiation. For the cultivation with PC12 cells, the washed electrically conductive hydrogels swollen in PBS were first rinsed for 10 min in 70% ethanol in order to kill any microorganisms and then washed in sterile PBS. After treatment with collagen type I (50 μg/ml, 1 ml/100 μl gel, Gibco™) in 20 mM sterile acetic acid for 2 h at room temperature, the gels were washed for 30 min in PBS. For NGF-β-loaded gels, an additional incubation with NGF-β1 (100 ng/ml, Sigma-Aldrich) in PBS with 1% BSA overnight took place. 50,000 cells per cm² of gel surface were then sown. The cells were sown and incubated in RPMI1640 (Gibco™) with an addition of 10% horse serum (Gibco™), 5% fetal calf serum (Gibco™) and 1% penicillin and streptamycin (Gibco™) in the medium. Depending on the cultivation condition, the pure medium was used, 100 ng/ml of NGF-β were added to the medium or nutrient-reduced medium (reduced) was used with only 1% of horse serum (Gibco™) and 0.5% of fetal calf serum. After incubation for 5 or 7 days with a medium change every 2 days, the cells were fixed for 20 min at room temperature in 4% PFA in PBS. For the imaging, the cells were colored with phalloidine (Abcam) and DAPI (Pierce) according to the manufacturer's instructions. Images were taken with a fluorescence microscope. The evaluation of the cell number and the cell circumference was carried out with FIJI/ImageJ using the watershed and analyze particles (cell number) and the skeletonize plug-in.

For all cultivation conditions, cell adhesion and cell proliferation were observed on the gels. In comparison with 7 to 5 days, a strong increase in the cell number occurred when the cell circumference on the hydrogels was reduced without addition of NGF-β (Table 6). This speaks for high cell proliferation. The addition of NGF-β into the medium leads to differentiation of the PC12 cells after already 5 days (Table 6). This was confirmed in an increase in the cell circumference by the growth of axon-like structures. Addition of NGF-β into the hydrogel but not into the medium leads to only a slight increase in the cell circumference with only a slight increase in the cell number (Table 6). Without stimulus, the amount of NGF-β released is too small to achieve complete differentiation of the cells. This is due to the high affinity of NGF-β to the hydrogel. If the nutrient content in the medium is reduced, an increased differentiation can be achieved. Accordingly, the cells have the ability to proliferate and differentiate on the hydrogels in all cultivation conditions, which confirms a high biocompatibility of the hydrogels.

### LIST OF REFERENCE NUMERALS

1 electrically conductive hydrogel material/hydrogel material
2 polymer network
3 anionically charged building blocks
4 crosslinker molecule
5 electrically conductive component/PEDOT
6 bioactive substance/bioactive substances/signal protein
7 voltage source

1.-22. (canceled)

23. A method for detecting and influencing an uptake and/or release of bioactive substances from a hydrogel material comprising,
a hydrogel material being formed as a polymer network from anionically charged building blocks and uncharged building blocks, said hydrogel material configured for affinity of bioactive substances by parameters defining the anionically charged building blocks,
said hydrogel material having an electrically conductive component, with an electrical resistance and electrical charge storage capacity depending on interacting with the hydrogel building blocks and binding of bioactive substances to the hydrogel material,
wherein the anionic charge of the hydrogel material and its affinity for bioactive substances is changed by the electrically conductive component by influencing an electric potential such that bringing the hydrogel material into contact with a biofluid, a change in electrical resistance and/or a change in charge storage capacity of the hydrogel material is detected and an uptake of bioactive substances into the hydrogel material or a release of bioactive substances from the hydrogel material into the biofluid occurs and is determined by the detected change in electrical resistance and/or the detected change in charge storage capacity, and wherein a concentration of bioactive substances in the biofluid and a concentration of bioactive substances in the hydrogel material is being influenced by an electrical potential acting on the hydrogel material.

24. The method according to claim 23, wherein prior to contacting the biofluid, the hydrogel material was loaded with a predetermined bioactive substance at a predetermined concentration for release of bioactive substances.

25. The method according to claim 24, wherein the polymer network is configured in its composition by at least three parameters defining the anionically charged building blocks, P0, P1, P2, P3,
wherein parameter P0 corresponds to a value from the number of ionized anionic groups, assuming a 30% ionization of all anionic groups, per unit volume of the hydrogel material swollen under physiological conditions, parameter P1 corresponds to a value from the number of strongly anionic groups, with an intrinsic pKS value smaller than 2.5, per unit volume of the hydrogel material swollen under physiological conditions, parameter P2 corresponds to a value of the number of strongly anionic groups, with an intrinsic pKS value smaller than 2.5, per repeating unit divided by the molar mass of the repeating unit, parameter 3 corresponds to a value for describing the amphiphilicity of the anionic building blocks, wherein an electrical resistance and/or an electrical charge storage capacity of the hydrogel material is predetermined by parameter values of a parameter configuration of the hydrogel material.

26. The method according to claim 23, wherein by detecting a binding of bioactive substances the hydrogel material incurs a change in the impedance of the hydrogel material measured at at least one frequency in the range of 0.1 Hz to 1 MHz.

27. The method according to claim 23, wherein the hydrogel material is acted upon by an electrical potential in the range from 1 mV to 1000 mV, for the uptake of bioactive substances, and wherein the hydrogel material is subjected to an electrical potential in the range from −1 mV to −1000 mV for the release of bioactive substances.

28. The method according to claim 27, wherein a constant electric current greater than 0 mA is applied to the hydrogel material for the uptake of bioactive substances, wherein the direction of the electric current flow is changed for the release of bioactive substances.

29. An electrically conductive hydrogel material comprising,
a polymer network formed of anionically charged building blocks and uncharged building blocks, the composition of which network is configurable on the basis of at least three parameters defining the anionically charged building blocks, selected from a group of parameters P0, P1, P2, P3,
said parameter P0 corresponding to a value from the number of ionized anionic groups, assuming a 30% ionization of all anionic groups, per unit volume of the hydrogel material swollen under physiological conditions,
said parameter P1 corresponding to a value from the number of strongly anionic groups, with an intrinsic pKS value smaller than 2.5, per unit volume of the hydrogel material swollen under physiological conditions,
said parameter P2 corresponding to a value from the number of strongly anionic groups, with an intrinsic pKS value smaller than 2.5, per repeating unit divided by the molar mass of the repeating unit,
said parameter P3 corresponding to a value describing the amphiphilicity of the anionic building blocks, and
an electrically conductive component incorporated in the polymer network, wherein an electrical conductivity, an electrical resistance and/or an electrical charge storage capacity of the hydrogel material is predeterminable by parameter values of a parameter configuration of the hydrogel material.

30. The electrically conductive hydrogel material according to claim 29, wherein the anionically charged building blocks are selected from a group consisting of poly(acrylic acid-co-4-acrylamidomethylbenzenesulfonic acid), poly(acrylic acid-co-acrylamidoethanesulfonic acid), poly(acrylic acid-co-acrylamidoethane hydrogen sulfate), poly(4-styrene sulfonic acid-co-maleic acid), sulfated glycosaminoglycans, selectively desulfated heparin derivatives, heparan sulfate, chondrotin sulfate, keratan sulfate and dermatan sulfate, and wherein the uncharged building blocks are amino group or thiol group containing polymers or crosslinker molecules having at least two amino groups or thiol groups, the charged and uncharged building blocks being crosslinked to form the polymer network, formed by activated carboxyl groups selected from one or more of the group of poly(acrylic acid-co-4-acrylamidomethylbenzenesulfonic acid), poly(acrylic acid-co-acrylamidoethanesulfonic acid), poly(acrylic acid-co-acrylamidoethane hydrogen sulfate), poly(4-styrenesulfonic acid-co-maleic acid), sulfated glycosaminoglycans, selectively desulfated heparin derivatives, heparan sulfate, chondrotin sulfate, keratan sulfate and dermatan sulfate with EDC/sulfo-NHS, are either directly crosslinked with the polymers containing amino groups, or the crosslinker molecules crosslinked with the at least two amino groups, under amide formation, or the activated carboxyl groups are functionalized by means of bifunctional crosslinker molecules, each containing an amino group, such that the subsequent polymer contains thiol groups or the crosslinker molecules with at least two thiol groups are crosslinked via a Michael-type addition.

31. The electrically conductive hydrogel material, according to claim 29, wherein an electrical impedance of the hydrogel material measured at a frequency of 0.01 Hz, is variable and in the range from 150Ω to 10 Ω.

32. The electrically conductive hydrogel material according to claim 29, wherein an electrical charge storage capacity of the hydrogel material is variable in a range from 900 mC/ml to 4000 mC/ml.

33. The. electrically conductive hydrogel material according to claim 29, wherein the electrically conductive component is a II-conjugated electrically conductive polymer or a polymer composition of polypyrrole, polyaniline, polythiophene and/or poly(3,4-ethylenedoxythiophene) (PEDOT).

34. The electrically conductive hydrogel material according to claim 30, wherein the polymers containing amino groups and thiol groups as uncharged building blocks are selected from the group consisting of polyethylene glycols (PEG), poly(2-oxazolines) (POX), polyvinylpyrrolidones (PVP), polyvinyl alcohols (PVA) and polyarylamides (PAM), and the amino group- or thiol group-containing crosslinker molecules are non-polymeric, bifunctional crosslinker molecules.

35. The electrically conductive hydrogel material according to claim 34, wherein the uncharged building blocks are polymers with conjugated enzymatically cleavable peptides having either lysine or cysteine as reactive amino acid in the peptide sequence.

36. The electrically conductive hydrogel material according to claim 35, wherein the enzymatically cleavable peptides are cleavable by one or more selected from the group consisting of matrix metalloproteases (MMPs), cathepsins, elastases, aureolysin and blood coagulation enzymes.

37. The electrically conductive hydrogel material according to claim 29, wherein bioactive and/or anti-adhesive molecules with an amino group or carboxyl group and/or cell-instructive peptides via lysine or cysteine are sequentially attached to the charged building blocks selected from the group consisting of poly(acrylic acid-co-4-acrylamidomethylbenzenesulfonic acid), poly(acrylicacid-co-acrylamidoethanesulfonic acid), poly(acrylicacid-co-acrylamidoethane hydrogen sulfate), poly(4-styrenesultfonic acid-co-maleic acid), sulfated glycosaminoglycans, selectively desulfated heparinderivatives, heparan sulfate, chondrotin sulfate, keratan sulfate, dermatan sulfate and derivatives thereof having groups capable of Michael-type addition and attached to the hydrogel network to form a covalent bond.

38. The electrically conductive hydrogel material according to claim 37, wherein the bioactive molecules are selected from the group of antibiotics, antiseptics and pharmaceutical agents.

39. The electrically conductive hydrogel material according to claim 29, wherein parameter P0 is preset at a value in a range from 0 to 80 μmol/ml, parameter P1 is preset at a value in a range from 0 to 150 μmol/ml, parameter P2 is preset at a value in a range from 0 to 10 mmol/(g/mol) and parameter P3 is preset at a value in a range from −7×10⁻³ to 7×10⁻³ A-2.

40. The electrically conductive hydrogel material according to claim 29, wherein the hydrogel material has a storage modulus of 0.2 kPa to 22 kPa.

41. A method of using an electrically conductive hydrogel material according to claim 29, comprising, managing an in vivo factor for controlling one or more diseases from the group consisting of angiogenesis, immune diseases, cancer diseases, diabetes, neurodegenerative diseases, Crohn's disease, ulcerative colitis, multiple sclerosis, asthma, rheumatoid arthritis or cutaneous wound healing and bone regeneration.

42. Use of an electrically conductive hydrogel material according to claim 29 for electrical stimulation of cells or tissues.

43. A method of using the electrically conductive hydrogel material according to any claim 29, comprising managing an in vivo factor for the control of angiogenesis, from the group of immune diseases, cancer diseases, diabetes, neurodegenerative diseases, Crohn's disease, ulcerative colitis, multiple sclerosis, asthma, rheumatoid arthritis or cutaneous wound healing and bone regeneration.

44. A method of using the electrically conductive hydrogel material according to claim 29, comprising for in vitro cell culture and organ culture of induced pluripotent stem (iPS) cells, non-iPS stem and progenitor cells, primary patient-derived cells, immortalized cell lines, as well as heart tissue, muscle tissue, kidney tissue, liver tissue and nerve tissue.

45. The method according to claim 27, wherein the hydrogel material is acted upon by an electrical potential in the range from 400 mV to-600 mV, for the release of bioactive substances.

46. The electrically conductive hydrogel material, according to claim 27, wherein the electrical impedance of the hydrogel material measured at a frequency of 0.01 Hz is at 30 Ω.

47. The electrical conductive hydrogel material of claim 32, wherein the parameter configuration is selected for a storage capacity of 2480 mC/ml.
